(19) United States
(12) Patent Application Publication
(10) Pub. No.: US 2020/0247856 A1
(43) Pub. Date: Aug. 6, 2020

(54) RECOMBINANT FOLLISTATIN-FC FUSION PROTEINS AND USE IN TREATING DUCHENNE MUSCULAR DYSTROPHY

(71) Applicant: Shire Human Genetic Therapies, Inc., Lexington, MA (US)

(72) Inventors: Andrea Iskenderian, Lexington, MA (US); Angela W. Norton, Lexington, MA (US); Chuan Shen, Lexington, MA (US); Clark Pan, Lexington, MA (US)

(21) Appl. No.: 16/082,070

(22) PCT Filed: Mar. 3, 2017
(86) PCT No.: PCT/US2017/020723

§ 371 Date: Sep. 4, 2018

Related U.S. Application Data
(60) Provisional application No. 62/303,954, filed on Mar. 4, 2016.

(51) Int. Cl. C07K 14/47; C07K 16/22; A61K 39/395; A61K 9/00; A61P 21/06

(52) U.S. Cl. CPC C07K 14/473; C07K 16/22; A61K 39/3955; A61K 38/00; A61P 21/06; C07K 2319/30; C07K 2317/92; A61K 9/0019

(57) ABSTRACT

The present invention provides, among other things, methods and compositions for treating muscular dystrophy, in particular, Duchenne muscular dystrophy (DMD). In some embodiments, a method according to the present invention includes administering to an individual who is suffering from or susceptible to DMD an effective amount of a recombinant follistatin fusion protein such that at least one symptom or feature of DMD is reduced in intensity, severity, or frequency, or has delayed onset.

### RELATED APPLICATIONS

This application claims the benefit of provisional application U.S. Ser. No. 62/303,954, filed Mar. 4, 2016, which is incorporated herein by reference in its entirety.

### BACKGROUND

Duchenne muscular dystrophy (DMD) is an X-linked recessive disorder affecting an estimated 1:3600 male births with an estimated 50,000 affected individuals worldwide. The disorder is marked by a progressive wasting of the muscles and affected children are wheelchair dependent by the time they reach 13 years of age. Affected individuals usually present with symptoms at 3 years of age with the median survival for such individuals being between 25 and 30 years of age. Respiratory failure due to diaphragmatic weakness and cardiomyopathy are common causes of death.

DMD is caused by a mutation in the dystrophin gene. The dystrophin gene is located on the X chromosome and codes for the protein dystrophin. Dystrophin protein is responsible for connecting the contractile machinery (actin-myosin complex) of a muscle fiber to the surrounding extracellular matrix through the dystroglycan complex. Mutations in the dystrophin gene result in either alteration or absence of the dystrophin protein and abnormal sarcolemma membrane function. While both males and females can carry a mutation in the dystrophin gene, females are rarely affected with DMD.

One characteristic of DMD is ischemia of the affected tissues. Ischemia is a restriction or decrease in blood supply to tissues or organs, causing a shortage of oxygen and nutrients need for cellular metabolism. Ischemia is generally caused by constriction or obstruction of blood vessels resulting in damage to or dysfunction of the tissue or organ. Treatment of ischemia is directed toward increasing the blood flow to the affected tissue or organ.

Presently, there is no cure for DMD. Several therapeutic avenues have been investigated including gene therapy and corticosteroid administration, however the need for alternatives for DMD patients still exists.

### SUMMARY OF THE INVENTION

The present invention provides, among other things, improved methods and compositions for the treatment of DMD based on administration of a recombinant follistatin-Fc fusion protein. The invention encompasses, inter alia, the unexpected observation that certain amino acid modifications in the follistatin polypeptide result in improved follistatin protein that specifically targets myostatin and activin A with high affinity and does not bind to non-target BMPs or heparin with meaningful affinity. It is contemplated that activation of Smad2/3 pathway by myostatin and activin A leads to inhibition of myogenic protein expression and as a result, myoblasts do not differentiate into muscle. Therefore, myostatin and activin are viable targets for stimulation of muscle regeneration. However, myostatin and activin antagonists including follistatin can bind bone morphogenetic proteins (BMPs) due to certain structural similarities. BMPs, especially, BMP-9 and BMP-10, are pivotal morphogenetic signals, orchestrating tissue architecture throughout the body. Inhibition of such BMPs may lead to undesired pathological conditions. Follistatin also binds to cell surface heparan-sulfate proteoglycans through a basic heparin-binding sequence (HBS) in the first of three FS domains. It is contemplated that inactivation, reduction or modulation of heparin binding may increase in vivo exposure and/or half-life of follistatin. Thus, the present invention provides improved follistatin that has longer half-life and is more potent for effective treatment of DMD.

In one aspect, the present invention provides recombinant follistatin polypeptides comprising an amino acid sequence at least 80% identical to SEQ ID NO: 1, SEQ ID NO: 2, SEQ ID NO: 3, SEQ ID NO: 4, or SEQ ID NO: 5, wherein the recombinant follistatin protein has a heparin binding domain (HBS), and wherein one or more amino acids within the HBS is substituted with an amino acid having a less positive charge in comparison to the substituted amino acid. In one embodiment, the one or more amino acids within the HBS are substituted with an amino acid having a neutral charge. In one embodiment, the one or more amino acids within the HBS are substituted with an amino acid having a negative charge. In one embodiment, the one or more comprises at least 1, 2, 3, 4, 5, 6, 7, 8, 9, or 10 amino acids. In one embodiment, the one or more comprises 3 amino acids. In one embodiment, the recombinant polypeptide has decreased heparin binding affinity in comparison to naturally occurring follistatin. In one embodiment, the recombinant follistatin protein does not bind to BMP-9 or BMP-10. In one embodiment, the recombinant follistatin protein has a sequence at least 80% identical to any one of SEQ ID NO: 12-40 or SEQ ID NO: 101-106.

In one aspect, the present invention provides recombinant follistatin polypeptides comprising an amino acid sequence at least 80% identical to SEQ ID NO:2, SEQ NO:4 or SEQ ID NO:5 and wherein the amino acids corresponding to positions 66 to 88 of SEQ ID NO:2, SEQ NO:4 or SEQ ID NO:5 are identical to any one of SEQ ID NO:42-67 or SEQ ID NO:111-116. In some embodiments, the amino acid sequence corresponding to positions 66 to 88 of SEQ ID NO: 2, SEQ ID NO: 4, or SEQ ID NO: 5 are identical to any one of SEQ ID NO: 58-67 or SEQ ID NO: 111-113. In some embodiments, the recombinant follistatin polypeptide is a hyperglycosylation mutant. In some embodiments, the amino acid sequence of the recombinant follistatin polypeptide is at least 90%, identical to SEQ ID NO:2, SEQ NO:4 or SEQ ID NO:5. In some embodiments, the amino acid sequence of the recombinant follistatin polypeptide is at least 95%, identical to SEQ ID NO:2, SEQ NO:4 or SEQ ID NO:5. In some embodiments, the amino acid sequence of the recombinant follistatin polypeptide is at least 98%, identical to SEQ ID NO:2, SEQ NO:4 or SEQ ID NO:5. In some embodiments, the amino acid sequence is of the recombinant follistatin polypeptide is 100% identical to SEQ ID NO:2, SEQ NO:4 or SEQ ID NO:5.

In one aspect, the present invention provides recombinant follistatin polypeptides comprising an amino acid sequence at least 80% identical to SEQ ID NO:2, SEQ NO:4 or SEQ ID NO:5 and comprising any one of the amino acid variations selected from the group consisting of C66S, C66A, G74N, K75E, K75N, K76A, K76D, K76S, K76E, C77S, C77T, R78E, R78N, N80T, K81A, K81D, K82A, K82D, K81E, K82T, K82E, K84E, P85T, R86N, V88E and V88T, or combinations thereof. In some embodiments, the amino acid sequence of the recombinant follistatin polypeptide is at least 90%, identical to SEQ ID NO:2, SEQ NO:4 or SEQ ID NO:5. In some embodiments, the amino acid sequence of the recombinant follistatin polypeptide is at least 95%, identical to SEQ ID NO:2, SEQ NO:4 or SEQ ID NO:5. In some embodiments, the amino acid sequence of the recombinant follistatin polypeptide is at least 98%, identical to SEQ ID NO:2, SEQ NO:4 or SEQ ID NO:5. In some embodiments, the amino acid sequence of the recombinant follistatin polypeptide is 100% identical to SEQ ID NO:2, SEQ NO:4 or SEQ ID NO:5.

In one aspect, the present invention provides recombinant follistatin polypeptides comprising an amino acid sequence selected from the group consisting of SEQ NO:12, SEQ ID NO:17-30 and SEQ ID NO:32-40.

In one aspect, the present invention provides recombinant follistatin fusion proteins comprising a recombinant follistatin polypeptide and an IgG Fc domain.

In one aspect, the present invention provides recombinant follistatin fusion proteins comprising a follistatin polypeptide and a human IgG Fc domain, wherein the recombinant follistatin polypeptide comprises an amino acid sequence at least 80% identical to SEQ ID NO:2, SEQ ID NO:4 or SEQ ID NO:5 and wherein the amino acids corresponding to positions 66 to 88 of SEQ ID NO:2, SEQ ID NO:4 or SEQ ID NO:5 are identical to SEQ ID NO:41, 42, 43 or 58. In some embodiments, the recombinant follistatin polypeptide comprises an amino acid sequence that is at least 90% identical to SEQ ID NO:2, SEQ ID NO:4 or SEQ ID NO:5. In some embodiments, the recombinant follistatin polypeptide comprises an amino acid sequence that is at least 95% identical to SEQ ID NO:2, SEQ ID NO:4 or SEQ ID NO:5. In some embodiments, the recombinant follistatin polypeptide comprises an amino acid sequence that is at least 98% identical to SEQ ID NO:2, SEQ ID NO:4 or SEQ ID NO:5. In some embodiments, the recombinant follistatin polypeptide comprises an amino acid sequence that is 100% identical to SEQ ID NO:2, SEQ ID NO:4 or SEQ ID NO:5.

In one aspect, the present invention provides recombinant follistatin fusion proteins comprising a follistatin polypeptide and an IgG Fc domain, wherein the follistatin polypeptide comprises an amino acid sequence selected from any one of the group consisting of SEQ ID NO:12, SEQ ID NO:13, and SEQ ID NO:15 to SEQ ID NO:40.

In some embodiments, the IgG Fc domain comprises an amino acid substitution wherein the amino acid substitution is selected from the group consisting of L234A, L235A, H433K, N434F, and combinations thereof, according to EU numbering.

In some embodiments, the IgG Fc domain comprises an amino acid sequence of SEQ ID NO:6 and wherein the amino acid sequence comprises an amino acid substitution selected from the group consisting of L234A, L235A, H433K, N434F, and combinations thereof, according to EU numbering.

In some embodiments, the IgG Fc domain comprises an amino acid sequence selected from the group consisting of SEQ ID NO:7 to SEQ ID NO:11. In some embodiments, the IgG Fc domain is a human IgG Fc domain. In some embodiments, the IgG Fc domain is an IgG1, IgG2, IgG3 or IgG4 Fc domain.

In one aspect, the present invention provides recombinant follistatin fusion proteins comprising an amino acid sequence of any one of SEQ ID NO:73 to SEQ ID NO:100.

In some embodiments, the recombinant follistatin fusion protein binds to myostatin with an affinity dissociation constant (K_{D}) of 1 to 100 pM. In some embodiments, the recombinant follistatin fusion protein binds activin A with an affinity dissociation constant (K_{D}) of 1 to 100 pM. In some embodiments, the recombinant follistatin fusion protein does not bind to bone morphogenic protein-9 (BMP-9) and/or bone morphogenic protein-10 (BMP-10) in the range of 0.2 nM to 25 nM. In some embodiments, the recombinant follistatin fusion protein binds to heparin with an affinity dissociation constant (K_{D}) of 0.1 to 200 nM. In some embodiments, the recombinant follistatin fusion protein binds to the Fc receptor with an affinity dissociation constant (K_{D}) of 25 to 400 nM.

In some embodiments, the recombinant follistatin fusion protein inhibits myostatin at an IC₅₀ of 0.1 to 10 nM. In some embodiments, the recombinant follistatin fusion protein inhibits activin at an IC₅₀ of 0.1 to 10 nM.

In one aspect, the present invention provides pharmaceutical compositions comprising a recombinant follistatin fusion protein and a pharmaceutically acceptable carrier.

In one aspect, the present invention provides a polynucleotide comprising a nucleotide sequence encoding the recombinant follistatin polypeptide.

In one aspect, the present invention provides a polynucleotide comprising a nucleotide sequence encoding the recombinant follistatin fusion protein. In some embodiments, an expression vector comprises the polynucleotide. In some embodiments, a host cell comprises a polynucleotide or an expression vector.

In one aspect, the present invention provides a method of making a recombinant follistatin fusion protein that specifically binds to myostatin and activin A by culturing the host cell.

In one aspect, the present invention provides a hybridoma cell producing a recombinant follistatin polypeptide or a recombinant follistatin fusion protein.

In one aspect, the present invention provides a method of treating Duchenne Muscular Dystrophy (DMD), the method comprising administering to a subject who is suffering from or susceptible to DMD an effective amount of the recombinant follistatin fusion protein or a pharmaceutical composition comprising the recombinant follistatin fusion protein, such that at least one symptom or feature of DMD is reduced in intensity, severity, or frequency, or has delayed onset.

In some embodiments, the method further comprises administering to the subject one or more additional therapeutic agents. In some embodiments the one or more additional therapeutic agents are selected from the group consisting of an anti-Flt-1 antibody or fragment thereof, edasalonexent, pamrevlumab prednisone, deflazacort, RNA modulating therapeutics, exon-skipping therapeutics and gene therapy.

In some embodiments, an effective amount of the recombinant follistatin fusion protein is administered parenterally. In some embodiments, the parenteral administration is selected from the group consisting of intravenous, intradermal, intrathecal, inhalation, transdermal (topical), intraocular, intramuscular, subcutaneous, transmucosal administration, or combinations thereof. In some embodiments, the parenteral administration is intravenous administration. In some embodiments, the parenteral administration is subcutaneous administration. In some embodiments, the recombinant follistatin fusion protein is administered daily, twice weekly, weekly, monthly or bimonthly. In some embodiments the recombinant follistatin fusion protein is administered twice weekly.

In some embodiments, the recombinant follistatin fusion protein is delivered to one or more skeletal muscles selected from Table 1. In some embodiments, the administration of the recombinant follistatin fusion protein results in an increase in the mass of a muscle relative to a control. In some embodiments, the muscle is one or more skeletal muscles selected from Table 1. In some embodiments, the muscle is selected from the group consisting of diaphragm, triceps, soleus, tibialis anterior, gastrocnemius, extensor digitorum longus, rectus abdominus, quadriceps, and combinations thereof. In some embodiments, the muscle is the gastrocnemius muscle. In some embodiments, the increase in the mass of the muscle is an increase of at least 10%, 20%, 30%, 40%, 50%, 60%, 70%, 80%, 90%, 100%, 150%, 200% or 500% relative to a control.

In some embodiments, the administration of the recombinant follistatin fusion protein results in muscle regeneration, increased muscle strength, increased flexibility, increased range of motion, increased stamina, reduced fatigability, increased blood flow, improved cognition, improved pulmonary function, inflammation inhibition, reduced muscle fibrosis, and/or reduced muscle necrosis.

In some embodiments, the at least one symptom or feature of DMD is selected from the group consisting of muscle wasting, muscle weakness, muscle fragility, muscle necrosis, muscle fibrosis, joint contracture, skeletal deformation, cardiomyopathy, impaired swallowing, impaired bowel and bladder function, muscle ischemia, cognitive impairment, behavioral dysfunction, socialization impairment, scoliosis, and impaired respiratory function.

In one aspect, the present invention provides methods for inhibiting myostatin in a subject, the method comprising administering to the muscle of a subject a composition comprising an effective amount of the recombinant follistatin fusion protein.

### BRIEF DESCRIPTION OF THE DRAWING

The drawings are for illustration purposes only, not for limitation.

FIGS. 1A and 1B show exemplary results illustrating serum PK profiles in CD-1 mice administered exemplary recombinant follistatin-Fc fusion proteins or FS315WT-hFc, a comparator protein.

FIG. 2 is a graph that shows forelimb grip strength in mdx mice treated with PBS vehicle, FS315K(76,81,82)E-mFc at 10 mg/kg, or ActRIIB-mFc at 3 mg/kg, in comparison to the grip strength in wild-type mice. Forelimb grip strength was measured after 11 weeks of dosing. The data show that there was a significant increase in forelimb grip strength of mdx mice treated with FS315K(76,81,82)E-mFc compared to the grip strength of animals treated with vehicle alone.

### DEFINITIONS

In order for the present invention to be more readily understood, certain terms are first defined below. Additional definitions for the following terms and other terms are set forth throughout the specification.

Affinity: As is known in the art, “affinity” is a measure of the tightness with a particular ligand binds to its partner. In some embodiments, the ligand or partner is a recombinant follistatin polypeptide. In some embodiments, the ligand or partner is a recombinant follistatin-Fc fusion protein. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Amelioration: As used herein, the term “amelioration” is meant the prevention, reduction or palliation of a state, or improvement of the state of a subject. Amelioration includes, but does not require complete recovery or complete prevention of a disease condition.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Bioavailability: As used herein, the term “bioavailability” generally refers to the percentage of the administered dose that reaches the blood stream of a subject.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Cardiac Muscle: As used herein, the term “cardiac muscle” refers to a type of involuntary striated muscle found in the walls of the heart, and particularly the myocardium.

Carrier or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g., phosphate-buffered saline), sterile saline solution, Ringer's solution or dextrose solution.

Dosage form: As used herein, the terms “dosage form” and “unit dosage form” refer to a physically discrete unit of a therapeutic protein (e.g., recombinant follistatin polypeptide or recombinant follistatin-Fc fusion protein) for the patient to be treated. Each unit contains a predetermined quantity of active material calculated to produce the desired therapeutic effect. It will be understood, however, that the total dosage of the composition will be decided by the attending physician within the scope of sound medical judgment.

Follistatin or recombinant follistatin: As used herein, the term “follistatin (FS)” or “recombinant follistatin” refers to any wild-type or modified follistatin proteins or polypeptides (e.g., follistatin proteins with amino acid mutations, deletions, insertions, and/or fusion proteins) that retain substantial follistatin biological activity unless otherwise specified.

Fc region: As used herein, the term “Fc region” refers to a dimer of two “Fc polypeptides”, each “Fc polypeptide” comprising the constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or all of the flexible hinge N-terminal to these domains. For IgG, “Fc polypeptide” comprises immunoglobulin domains Cgamma2 (Cy2) and Cgamma3 (Cy3) and the lower part of the hinge between Cgamma1 (Cy1) and Cy2. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, Va.). For IgA, Fc polypeptide comprises immunoglobulin domains Calpha2 (Ca2) and Calpha3 (Ca3) and the lower part of the hinge between Calpha1 (Ca1) and Ca2. An Fc region can be synthetic, recombinant, or generated from natural sources such as IVIG.

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Fusion protein: As used herein, the term “fusion protein” or “chimeric protein” refers to a protein created through the joining of two or more originally separate proteins, or portions thereof. In some embodiments, a linker or spacer will be present between each protein. A non-limiting example of a fusion protein is an Fc-fusion protein. A non-limiting example of a fusion protein is a follistatin-Fc fusion protein.

Half-Life: As used herein, the term “half-life” is the time required for a quantity such as protein concentration or activity to fall to half of its value as measured at the beginning of a time period.

Hypertrophy: As used herein the term “hypertrophy” refers to the increase in volume of an organ or tissue due to the enlargement of its component cells.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Inhibition: As used herein, the terms “inhibition,” “inhibit” and “inhibiting” refer to processes or methods of decreasing or reducing activity and/or expression of a protein or a gene of interest. Typically, inhibiting a protein or a gene refers to reducing expression or a relevant activity of the protein or gene by at least 10% or more, for example, 20%, 30%, 40%, or 50%, 60%, 70%, 80%, 90% or more, or a decrease in expression or the relevant activity of greater than 1-fold, 2-fold, 3-fold, 4-fold, 5-fold, 10-fold, 50-fold, 100-fold or more as measured by one or more methods described herein or recognized in the art.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

K_{D}: As used herein, the term “K_{D}”, as used herein, is intended to refer to the dissociation constant, which is obtained from the ratio of Kd to Ka Kd/Ka) and is expressed as a molar concentration (M). K_{D} values for a ligand can be determined using methods well established in the art. A preferred method for determining the K_{D} of an ligand is by using surface plasmon resonance, preferably using a biosensor system such as a BIAcore® system.

Linker: As used herein, the term “linker” refers to, in a fusion protein, an amino acid sequence other than that appearing at a particular position in the natural protein and is generally designed to be flexible or to interpose a structure, such as an α-helix, between two protein moieties. A linker is also referred to as a spacer. A linker or a spacer typically does not have biological function on its own.

Pharmaceutically acceptable: As used herein, the term “pharmaceutically acceptable” refers to substances that, within the scope of sound medical judgment, are suitable for use in contact with the tissues of human beings and animals without excessive toxicity, irritation, allergic response, or other problem or complication, commensurate with a reasonable benefit/risk ratio.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used inter-changeably.

Prevent: As used herein, the term “prevent” or “prevention”, when used in connection with the occurrence of a disease, disorder, and/or condition, refers to reducing the risk of developing the disease, disorder and/or condition. See the definition of “risk.”

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Risk: As will be understood from context, a “risk” of a disease, disorder, and/or condition comprises a likelihood that a particular individual will develop a disease, disorder, and/or condition (e.g., muscular dystrophy). In some embodiments, risk is expressed as a percentage. In some embodiments, risk is from 0, 1, 2, 3, 4, 5, 6, 7, 8, 9, 10, 20, 30, 40, 50, 60, 70, 80, 90 and up to 100%. In some embodiments risk is expressed as a risk relative to a risk associated with a reference sample or group of reference samples. In some embodiments, a reference sample or group of reference samples have a known risk of a disease, disorder, condition and/or event (e.g., muscular dystrophy). In some embodiments a reference sample or group of reference samples are from individuals comparable to a particular individual. In some embodiments, relative risk is 0, 1, 2, 3, 4, 5, 6, 7, 8, 9, 10, or more.

Striated muscle: As used herein, the term “striated muscle” refers to multinucleated muscle tissue with regular arrangement of their intracellular contractile units, sarcomeres, leading to the appearance of striations using microscopy and under voluntary control. Typically, striated muscle can be cardiac muscle, skeletal muscle, and Branchiomeric muscles.

Smooth muscle: As used herein, the term “smooth muscle” refers to involuntarily controlled, non-striated muscle, including unitary and multi-unit muscle.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding residues are homologous over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Surface plasmon resonance: as used herein, refers to an optical phenomenon that allows for the analysis of specific binding interactions in real-time, for example through detection of alterations in protein concentrations within a biosensor matrix, such as by using a BIAcore® system (Pharmacia Biosensor AB, Uppsala, Sweden and Piscataway, N.J.). For further descriptions, see Jonsson, U., et al. (1993) Ann. Biol. Clin. 51: 19-26; Jonsson, U., et al. (1991) Biotechniques 11:620-627; Johnsson, B., et al. (1995) J. Mol. Recognit. 8: 125-131; and Johnnson, B., et al. (1991) Anal. Biochem. 198:268-277.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, condition, or event (for example, DMD) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, condition, and/or event (5) having undergone, planning to undergo, or requiring a transplant. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Target tissues: As used herein, the term “target tissues” refers to any tissue that is affected by a disease to be treated such as Duchenne muscular dystrophy (DMD). In some embodiments, target tissues include those tissues that display disease-associated pathology, symptom, or feature, including but not limited to muscle wasting, skeletal deformation, cardiomyopathy, and impaired respiratory function.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION OF CERTAIN EMBODIMENTS

The present invention provides, among other things, methods and compositions for treating muscular dystrophy, including Duchenne muscular dystrophy (DMD) and/or Becker muscular dystrophy, based on follistatin as a protein therapeutic. In some embodiments, the present invention provides methods of treating DMD including administering to an individual who is suffering from or susceptible to DMD an effective amount of a recombinant follistatin protein or a recombinant follistatin-Fc fusion protein such that at least one symptom or feature of DMD is reduced in intensity, severity, or frequency, or has delayed onset.

Various aspects of the invention are described in detail in the following sections. The use of sections is not meant to limit the invention. Each section can apply to any aspect of the invention. In this application, the use of “or” means “and/or” unless stated otherwise.

### Duchenne Muscular Dystrophy (DMD)

DMD is a disease characterized by progressive deterioration of muscles and loss of muscle related functions throughout the body. It is contemplated that the present invention provides methods and compositions for regenerating muscle and treating fibrosis, inflammation and other symptoms or features associated with DMD and other muscular dystrophies in various muscle tissues. In some embodiments, use of provided methods and compositions in a subject result in a decrease fibrosis and/or necrosis in that subject.

Muscle Tissues

There are two major types of muscle tissue in an animal—striated muscle and smooth muscle. As used herein, the term “striated muscle” refers to muscle tissues containing repeating sarcomeres. Striated muscle tends to be under voluntary control and attached to the skeleton, though there are some exceptions, such as cardiac muscle, which has several properties of striated muscle, but is not under voluntary control. Generally, striated muscle allows for voluntary movement of the body and includes the major muscle groups including the quadriceps, gastrocnemius, biceps, triceps, trapezius, deltoids, and many others. Striated muscle tends to be very long and, many striated muscles are able to function independently. Some striated muscle, however, is not attached to the skeleton, including those in the mouth, anus, heart, and upper portion of the esophagus.

Smooth muscle, on the other hand, has very different structure. Rather than a series of long muscles with separate skeletal attachments, smooth muscle tends to be organized into continuous sheets with mechanical linkages between smooth muscle cells. Smooth muscle is often located in the walls of hollow organs and is usually not under voluntary control. Smooth muscles lining a particular organ must bear the same load and contract concurrently. Smooth muscle functions, at least in part, to handle changes in load on hollow organs caused by movement and/or changes in posture or pressure. This dual role means that smooth muscle must not only be able to contract like striated muscle, but also that it must be able to contract tonically to maintain organ dimensions against sustained loads. Examples of smooth muscles are those lining blood vessels, bladder, gastrointestinal track such as rectum.

The strength of a muscle depends on the number and sizes of the muscle's cells and on their anatomic arrangement. Increasing the diameter of a muscle fiber either by the increase in size of existing myofibrils (hypertrophy) and/or the formation of more muscle cells (hyperplasia) will increase the force-generating capacity of the muscle.

Muscles may also be grouped by location or function. In some embodiments, a recombinant follistatin protein is targeted to one or more muscles of the face, one or more muscles for mastication, one or more muscles of the tongue and neck, one or more muscles of the thorax, one or more muscles of the pectoral girdle and arms, one or more muscles of the arm and shoulder, one or more ventral and dorsal forearm muscles, one or more muscles of the hand, one or more muscles of the erector spinae, one or more muscles of the pelvic girdle and legs, and/or one or more muscles of the foreleg and foot.

In some embodiments, muscles of the face include, but are not limited to, intraocular muscles such as ciliary, iris dilator, iris sphincter; muscles of the ear such as auriculares, temporoparietalis, stapedius, tensor tympani; muscles of the nose such as procerus, nasalis, dilator naris, depressor septi nasi, levator labii superioris alaeque nasi; muscles of the mouth such as levator anguli oris, depressor anguli oris, orbicularis oris, Buccinator, Zygomaticus Major and Minor, Platysma, Levator Labii Superioris, Depressor Labii Inferioris, Risorius, Mentalis, and/or Corrugator Supercilii.

In some embodiments, muscles of mastication include, but are not limited to, Masseter, Temporalis, Medial Pterygoid, Lateral Pterygoid. In some embodiments, muscles of the tongue and neck include, but are not limited to, Genioglossus, Styloglossus, Palatoglossus, Hyoglossus, Digastric, Stylohyoid, Mylohyoid, Geniohyoid, Omohyoid, Sternohyoid, Sternothyroid, Thyrohyoid, Sternocleidomastoid, Anterior Scalene, Middle Scalene, and/or Posterior Scalene.

In some embodiments, muscles of the thorax, pectoral girdle, and arms include, but are not limited to, Subclavius Pectoralis major, Pectoralis minor, Rectus abdominis, External abdominal oblique, Internal abdominal oblique, Transversus Abdominis, Diaphragm, External Intercostals, Internal Intercostals, Serratus Anterior, Trapezius, Levator Scapulae, Rhomboideus Major, Rhomboideus Minor, Latissimus dorsi, Deltoid, subscapularis, supraspinatus, infraspinatus, Teres major, Teres minor, and/or Coracobrachialis.

In some embodiments, muscles of the arm and shoulder include, but are not limited to, Biceps brachii-Long Head, Biceps brachii-Short Head, Triceps brachii-Long Head, Triceps brachii Lateral Head, Triceps brachii-Medial Head, Anconeus, Pronator teres, Supinator, and/or Brachialis.

In some embodiments, muscles of the ventral and dorsal forearm include, but are not limited to, Brachioradialis, Flexor carpi radialis, Flexor carpi ulnaris, Palmaris longus, Extensor carpi ulnaris, Extensor carpi radialis longus, Extensor carpi radialis brevis, Extensor digitorum, Extensor digiti minimi.

In some embodiments, muscles of the hand include, but are not limited to intrinsic muscles of the hand such as thenar, abductor pollicis brevis, flexor pollicis brevis, opponens pollicis, hypothenar, abductor digiti minimi, the flexor digiti minimi brevis, opponens digiti minimi, palmar interossei, dorsal interossei and/or lumbricals.

In some embodiments, muscles of the erector spinae include, but are not limited to, cervicalis, spinalis, longissimus, and/or iliocostalis.

In some embodiments, muscles of the pelvic girdle and the legs include, but are not limited to, Psoas Major, Iliacus, quadratus femoris, Adductor longus, Adductor brevis, Adductor magnus, Gracilis, Sartorius, Quadriceps femoris such as, rectus femoris, vastus lateralis, vastus medialis, vastus intermedius, Gastrocnemius, Fibularis (Peroneus) Longus, Soleus, Gluteus maximus, Gluteus medius, Gluteus minimus, Hamstrings: Biceps Femoris: Long Head, Hamstrings: Biceps Femoris: Short Head, Hamstrings: Semitendinosus, Hamstrings: Semimembranosus, Tensor fasciae latae, Pectineus, and/or Tibialis anterior.

In some embodiments, muscles of the foreleg and foot include, but are not limited to, Extensor digitorum longus, Extensor hallucis longus, peroneus brevis, plantaris, Tibialis posterior, Flexor hallucis longus, extensor digitorum brevis, extensor hallucis brevis, Abductor hallucis, flexor hallucis brevis, Abductor digiti minimi, flexor digiti minimi, opponens digiti minimi, extensor digitorum brevis, lumbricales of the foot, Quadratus plantae or flexor accessorius, flexor digitorum brevis, dorsal interossei, and/or plantar interossei.

Exemplary muscle targets are summarized in Table 1.

TABLE 1
Muscle Targets
ORBICULARIS OCULI

Intraocular: ciliary, iris dilator, iris sphincter
Ear: auriculares, temporoparietalis, stapedius, tensor tympani
Nose: procerus, nasalis, dilator naris, depressor septi nasi, levator labii superioris alaeque nasi
Mouth: levator anguli oris, depressor anguli oris, orbicularis oris

| | | | |
|---|---|---|---|
| Buccinator | Zygomaticus Major | Platysma | Levator Labii |
| | and Minor | | Superioris |
| Depressor Labii | Risorius | Mentalis | Corrugator Supercilii |
| Inferioris | | | |
| Anconeus | Pronator teres | Supinator | Brachialis |

MUSCLES OF MASTICATON

| | | | |
|---|---|---|---|
| Masseter | Temporalis | Medial Pterygoid | Lateral Pterygoid |

MUSCLES OF THE TONGUE AND NECK

| | | | |
|---|---|---|---|
| Genioglossus | Styloglossus | Palatoglossus | Hyoglossus |
| Digastric | Stylohyoid | Mylohyoid | Geniohyoid |
| Omohyoid | Sternohyoid | Sternothyroid | Thyrohyoid |
| Sternocleidomastoid | Anterior Scalene | Middle Scalene | Posterior Scalene |

MUSCLES OF THE THORAX, PECTORAL GIRDLE AND ARMS

| | | | |
|---|---|---|---|
| Subclavius | Pectoralis major | Pectoralis minor | Rectus abdominis |
| External abdominal | Internal abdominal | Transversus | Diaphragm |
| oblique | oblique | Abdominis | |
| External Intercostals | Internal Intercostals | Serratus Anterior | Trapezius |
| Levator Scapulae | Rhomboideus Major | Rhomboideus Minor | Latissimus dorsi |
| Deltoid | subscapularis | supraspinatus | infraspinatus |
| Teres major | Teres minor | Coracobrachialis | |

ARM AND SHOULDER

| | | | |
|---|---|---|---|
| Biceps brachii-Long | Biceps brachii-Short | Triceps brachii-Long | Triceps brachii-Lateral |
| Head | Head | Head | Head |
| Triceps brachii-Medial | Anconeus | Pronator teres | Supinator |
| Head | | | |
| Brachialis | | | |

FOREARM MUSCLES: Ventral and Dorsal

| | | | |
|---|---|---|---|
| Brachioradialis | Flexor carpi radialis | Flexor carpi ulnaris | Palmaris longus |
| Extensor carpi ulnaris | Extensor carpi | Extensor carpi | Extensor digitorum |
| | radialis longus | radialis brevis | |
| Extensor digiti minimi | erector spinae: | erector spinae: | erector spinae: |
| | cervicalis | spinalis | longissimus |
| erector spinae: | | | |
| iliocostalis | | | |

Intrinsic Muscles of the Hand: thenar, abductor pollicis brevis, flexor pollicis brevis, and the
opponens pollicis
Intrinsic Muscles of the Hand: hypothenar, abductor digiti minimi, the flexor digiti minimi
brevis, and the opponens digiti minimi
Intrinsic Muscles of the Hand: palmar interossei, dorsal interossei and lumbricals

MUSCLES OF THE PELVIC GIRDLE AND THE LEGS

| | | | |
|---|---|---|---|
| Iliopsoas: Psoas Major | Iliopsoas: Iliacus | quadratus femoris | Adductor longus |
| Adductor brevis | Adductor magnus | Gracilis | Sartorius |
| Quadriceps femoris: | Quadriceps femoris: | Quadriceps femoris: | Quadriceps femoris: |
| rectus femoris | vastus lateralis | vastus medialis | vastus intermedius |
| Gastrocnemius | Fibularis (Peroneus) | Soleus | Gluteus maximus |
| | Longus | | |
| Gluteus medius | Gluteus minimus | Hamstrings: Biceps | Hamstrings: Biceps |
| | | Femoris: Long Head | Femoris: Short Head |
| Hamstrings: | Hamstrings: | Tensor fasciae latae | Pectineus |
| Semitendinosus | Semimembranosus | | |
| Tibialis anterior | | | |

MUSCLES OF THE FORELEG AND FOOT

| | | | |
|---|---|---|---|
| Extensor digitorum | Extensor hallucis | peroneus brevis | plantaris |
| longus | longus | | |
| Tibialis posterior | Flexor hallucis longus | extensor digitorum | extensor hallucis |
| | | brevis | brevis |
| Abductor hallucis | flexor hallucis brevis | Abductor digiti minimi | flexor digiti minimi |
| opponens digiti minimi | extensor digitorum | lumbricales of the | Quadratus plantae |
| | brevis | foot | or flexor |
| | | | accessorius |
| Flexor digitorum brevis | dorsal interossei | plantar interossei | |

Muscular Dystrophy

Muscular dystrophies are a group of inherited disorders that cause degeneration of muscle, leading to weak and impaired movements. A central feature of all muscular dystrophies is that they are progressive in nature. Muscular dystrophies include, but are not limited to: Duchenne muscular dystrophy (DMD), Becker muscular dystrophy, Emery-Dreifuss muscular dystrophy, facioscapulohumeral muscular dystrophy, limb-girdle muscular dystrophies, and myotonic dystrophy Types 1 and 2, including the congenital form of myotonic dystrophy Type 1. Symptoms may vary by type of muscular dystrophy with some or all muscles being affected. Exemplary symptoms of muscular dystrophies include delayed development of muscle motor skills, difficulty using one or more muscle groups, difficulty swallowing, speaking or eating, drooling, eyelid drooping, frequent falling, loss of strength in a muscle or group of muscles as an adult, loss in muscle size, problems walking due to weakness or altered biomechanics of the body, muscle hypertrophy, muscle pseudohypertrophy, fatty infiltration of muscle, replacement of muscle with non-contractile tissue (e.g., muscle fibrosis), muscle necrosis, and/or cognitive or behavioral impairment/mental retardation.

While there are no known cures for muscular dystrophies, several supportive treatments are used which include both symptomatic and disease modifying therapies. Corticosteroids, physical therapy, orthotic devices, wheelchairs, or other assistive medical devices for ADLs and pulmonary function are commonly used in muscular dystrophies. Cardiac pacemakers are used to prevent sudden death from cardiac arrhythmias in myotonic dystrophy. Anti-myotonic agents which improve the symptoms of myotonia (inability to relax) include mexilitine, and in some cases phenytoin, procainamide and quinine.

Duchenne Muscular Dystrophy

Duchenne muscular dystrophy (DMD) is a recessive X-linked form of muscular dystrophy which results in muscle degeneration and eventual death. DMD is characterized by weakness in the proximal muscles, abnormal gait, pseudohypertrophy in the gastrocnemius (calf) muscles, and elevated creatine kinase (CK). Many DMD patients are diagnosed around the age of 5, when symptoms/signs typically become more obvious. Affected individuals typically stop walking around age 10-13 and die in or before their mid to late 20's due to cardiorespiratory dysfunction.

The disorder DMD is caused by a mutation in the dystrophin gene, located on the human X chromosome, which codes for the protein dystrophin, an important structural component within muscle tissue that provides structural stability to the dystroglycan complex (DGC) of the cell membrane. Dystrophin links the internal cytoplasmic actin filament network and extracellular matrix, providing physical strength to muscle fibers. Accordingly, alteration or absence of dystrophin results in abnormal sarcolemmal membrane tearing and necrosis of muscle fibers. While persons of both sexes can carry the mutation, females rarely exhibit severe signs of the disease.

A primary symptom of DMD is muscle weakness associated with muscle wasting with the voluntary muscles being first affected typically, especially affecting the muscles of the hips, pelvic area, thighs, shoulders, and calf muscles. Muscle weakness also occurs in the arms, neck, and other areas. Calves are often enlarged. Signs and symptoms usually appear before age 6 and may appear as early as infancy. Other physical symptoms include, but are not limited to, delayed ability to walk independently, progressive difficulty in walking, stepping, or running, and eventual loss of ability to walk (usually by the age of 15); frequent falls; fatigue; difficulty with motor skills (running, hopping, jumping); increased lumbar lordosis, leading to shortening of the hip-flexor muscles; contractures of achilles tendon and hamstrings impairing functionality because the muscle fibers shorten and fibrosis occurs in connective tissue; muscle fiber deformities; pseudohypertrophy (enlargement) of tongue and calf muscles caused by replacement of muscle tissue by fat and connective tissue; higher risk of neurobehavioral disorders (e.g., ADHD), learning disorders (dyslexia), and non-progressive weaknesses in specific cognitive skills (in particular short-term verbal memory); skeletal deformities (including scoliosis in some cases).

### Recombinant Follistatin Proteins

As used herein, recombinant follistatin proteins suitable for the present invention include any wild-type and modified follistatin proteins (e.g., follistatin proteins with amino acid mutations, deletions, insertions, and/or fusion proteins) that retain substantial follistatin biological activity. Typically, a recombinant follistatin protein is produced using recombinant technology. However, follistatin proteins (wild-type or modified) purified from natural resources or synthesized chemically can be used according to the present invention. Typically, a suitable recombinant follistatin protein or a recombinant follistatin fusion protein has an in vivo half-life of or greater than about 12 hours, 18 hours, 24 hours, 36 hours, 2 days, 2.5 days, 3 days, 3.5 days, 4 days, 4.5 days, 5 days, 5.5 days, 6 days, 6.5 days, 7 days, 7.5 days, 8 days, 8.5 days, 9 days, 9.5 days, or 10 days. In some embodiments, a recombinant follistatin protein has an in vivo half-life of between 0.5 and 10 days, between 1 day and 10 days, between 1 day and 9 days, between 1 day and 8 days, between 1 day and 7 days, between 1 day and 6 days, between 1 day and 5 days, between 1 day and 4 days, between 1 day and 3 days, between 2 days and 10 days, between 2 days and 9 days, between 2 days and 8 days, between 2 days and 7 days, between 2 days and 6 days, between 2 days and 5 days, between 2 days and 4 days, between 2 day and 3 days, between 2.5 days and 10 days, between 2.5 days and 9 days, between 2.5 days and 8 days, between 2.5 days and 7 days, between 2.5 days and 6 days, between 2.5 days and 5 days, between 2.5 days and 4 days, between 3 days and 10 days, between 3 days and 9 days, between 3 days and 8 days, between 3 days and 7 days, between 3 days and 6 days, between 3 days and 5 days, between 3 days and 4 days, between 3.5 days and 10 days, between 3.5 days and 9 days, between 3.5 days and 8 days, between 3.5 days and 7 days, between 3.5 days and 6 days, between 3.5 days and 5 days, between 3.5 days and 4 days, between 4 days and 10 days, between 4 days and 9 days, between 4 days and 8 days, between 4 days and 7 days, between 4 days and 6 days, between 4 days and 5 days, between 4.5 days and 10 days, between 4.5 days and 9 days, between 4.5 days and 8 days, between 4.5 days and 7 days, between 4.5 days and 6 days, between 4.5 days and 5 days, between 5 days and 10 days, between 5 days and 9 days, between 5 days and 8 days, between 5 days and 7 days, between 5 days and 6 days, between 5.5 days and 10 days, between 5.5 days and 9 days, between 5.5 days and 8 days, between 5.5 days and 7 days, between 5.5 days and 6 days, between 6 days and 10 days, between 7 days and 10 days, between 8 days and 10 days, between 9 days and 10 days.

Follistatin (FS) was first isolated from follicular fluid, as a protein factor capable of suppressing pituitary cell follicle stimulating hormone (FSH) secretion. FS exerts its influence over FSH at least in part through the binding and neutralization of activin.

There are at least three isoforms of FS: FS288, FS303 and FS315 (Table 3). The full-length FS315 protein comprises an acidic 26-residue C-terminal tail encoded by exon 6 (SEQ ID NO:2, C-terminal tail is single underlined). In some instances the FS315 isoform may comprise a signal sequence (SEQ ID NO:1, signal sequence is designated in bold and italic). The FS288 isoform is produced through alternative splicing at the C-terminus and thus, ends with exon 5 (SEQ ID NO:5). The follistatin proteins have a distinctive structure comprised of a 63 amino acid N-terminal region containing hydrophobic residues important for activin binding, with the major portion of the protein (residues 64-288, for example as shown in SEQ ID NO:2) comprising three 10-cysteine FS domains of approximately 73-75 amino acids each. These 10-cysteine domains, from N-terminus to C-terminus, are referred to as domain 1, domain 2 and domain 3, respectively (i.e., FSD1, FSD2 and FSD3). FS288 tends to be tissue-bound due to the presence of a heparin binding domain, while FS315 tends to be a circulating form, potentially because the heparin binding domain is masked by the extended C-terminus. FS303 (SEQ ID NO:4) is thought to be produced by proteolytic cleavage of the C-terminal domain from FS315. In some instances the FS303 isoform may comprise a signal sequence (SEQ ID NO:3, signal sequence is designated in bold and italic). FS303 has an intermediate level of cell surface binding between that of FS288 and FS315.

The heparin binding domain or sequence (e.g., HBS) comprises amino acids corresponding to residues 75-86 of FS315 and is within the FSD1, as shown, for example, in SEQ ID NO:2. The HBS is designated by double underline. The FS303 and FS288 proteins also comprise an HBS at the corresponding amino acids (also designated by double underline). Mutation, deletion or substitution of amino acids within this region can reduce or abolish heparin binding and thereby reduce clearance and improve half-life of therapeutic follistatin-Fc fusion proteins.

In some embodiments, substitution of at least one or more amino acids within the HBS, with an amino acid that has a less positive charge, results in the recombinant follistatin protein having decreased heparin binding affinity. In some embodiments, substitution of at least one or more amino acids within the HBS, with an amino acid that has a more neutral or negative change, results in the recombinant follistatin protein having decreased heparin binding affinity. In some embodiments, substitution with an amino acid that has a reduced charge in comparison to the original amino acid results in the recombinant follistatin protein having decreased heparin binding affinity. In some embodiments, 1, 2, 3, 4, 5, 6, 7, 8, 9, or 10 substitutions of amino acids present in the HBS with amino acids that have a less positive charge, a neutral charge, a more negative charge, or a reduced charge results in the recombinant follistatin protein having decreased heparin binding affinity. In some embodiments, 1, 2, or 3 substitutions of amino acids present in the HBS with amino acids that have a less positive charge, a neutral charge, a more negative charge, or a reduced charge results in the recombinant follistatin protein having decreased heparin binding affinity. In some embodiments, substituting more than one amino acid in the HBS with less positively charged amino acids, neutral amino acids, a negatively charged amino acid, or a reduced charge amino acid results in progressively decreased heparin binding corresponding to the amount of amino acid substitutions made. For example, substituting 3 amino acids in the HBS with amino acids that have a less positive charge, a neutral charge, a more negative charge, or a reduced charge amino acid results in less heparin binding by the recombinant follistatin protein in comparison to substituting only 2 amino acids in the HBS with amino acids that have a less positive charge, a neutral charge, a more negative charge, or a reduced charge amino acid. As another example, substituting 2 amino acids in the HBS with amino acids that have a less positive charge, a neutral charge, a more negative charge, or a reduced charge amino acid results in less heparin binding by the recombinant follistatin protein in comparison to substituting only 1 amino acid in the HBS with an amino acid with a less positive charge, a neutral charge, a more negative charge, or a reduced charge amino acid.

One of skill in the art will recognize that certain amino acids are less positively charged, are neutral, are negatively charged or have a reduced charge in comparison to other amino acids. Amino acids can be separated based on net charge as indicated by an amino acid's isoelectric point. The isoelectric point is the pH at which the average net charge of the amino acid molecule is zero. When pH>pI, an amino acid has a net negative charge, and when the pH<pI, an amino acid has a net positive charge. In some embodiments, the measured pI value for a recombinant follistatin protein is between about 3 and 9 (e.g. 3.0, 3.5, 4.0, 4.5, 5.0, 5.5, 6.0, 6.5, 7.0, 7.5, 8.0, 8.1, 8.2, 8.3, 8.4, 8.5, and 9) and any values in between. In some embodiments, the measured pI value for a recombinant follistatin protein is between about 4 and 7 (e.g. 4.0, 4.5, 5.0, 5.5, 6.0, 6.5, 7.0), and any values in between. Exemplary isoelectric points of amino acids are shown in Table 2 below. Generally Amino acids with positive electrically charged side chains include, for example, Arginine (R), Histidine (H), and Lysine (K). Amino acids with negative electrically charged side chains include, for example, Aspartic Acid (D) and Glutamic Acid (E). Amino acids with polar properties include, for example, Serine (S), Threonine (T), Asparagine (N), Glutamine (Q), and Cysteine (C), Tyrosine (Y) and Tryptophan (W). Non-polar amino acids include, for example, Alanine (A), Valine (V), Isoleucine (I), Leucine (L), Methionine (M), Phenylalanine (F), Glycine (G) and Proline (P).

In some embodiments, point mutations in the HBS include one or more substitutions of one or more lysine (K) residues in the HBS. For example, one or more (e.g. 1, 2, 3, 4, 5) lysine residues are substituted for another amino acid in the HBS of the follistatin polypeptide. The HBS comprises amino acids corresponding to residues 75-86 of FS315, namely, residues KKCRMNKKNKPR. In some embodiments, substituting one or more negatively charged amino acids, for example Glutamic Acid (E) and/or Aspartic Acid (D), for the lysine (K) amino acid results in a change of the overall charge of the recombinant follistatin polypeptide, known as a pI shift. In some embodiments, a change in the overall charge of the follistatin molecule improves in-vivo clearance and half-life. In one embodiment, a change in the overall charge of the recombinant follistatin polypeptide slows in vivo clearance. In some embodiments, substituting one or more negatively charged amino acids, for example Glutamic Acid (E) and/or Aspartic Acid (D), for one or more lysine (K) amino acid results in a change of the overall charge of the recombinant follistatin molecule. In some embodiments, substituting one or more negatively charged amino acids, for example Glutamic Acid (E) and/or Aspartic Acid (D), for one or more lysine (K) amino acid results in a decrease in the amounts of high molecular weight species during expression of the recombinant follistatin polypeptide. In some embodiments, substituting one or more negatively charged amino acids, for example Glutamic Acid (E) and/or Aspartic Acid (D), for one or more lysine (K) amino acid results in increased expression of the recombinant follistatin polypeptide.

TABLE 2
Amino acid isoelectric points

| Amino Acid | One Letter Abbreviation | pI (isoelectric point) |
|---|---|---|
| Alanine | A | 6.0 |
| Arginine | R | 10.76 |
| Asparagine | N | 5.41 |
| Aspartic Acid | D | 2.77 |
| Cysteine | C | 5.07 |
| Glutamic Acid | E | 3.22 |
| Glutamine | Q | 5.65 |
| Glycine | G | 5.97 |
| Histidine | H | 7.59 |
| Isoleucine | I | 6.02 |
| Leucine | L | 5.98 |
| Lysine | K | 9.74 |
| Methionine | M | 5.74 |
| Phenylalanine | F | 5.48 |
| Proline | P | 6.30 |
| Serine | S | 5.58 |
| Threonine | T | 5.60 |
| Tryptophan | W | 5.89 |
| Tyrosine | Y | 5.66 |
| Valine | V | 5.96 |

It has been shown that FS inhibits both myostatin and activin in vitro and that this inhibition can lead to muscle hypertrophy in vivo in mice (Lee et al., Regulation of Muscle Mass by Follistatin and Activins, (2010), Mol. Endocrinol., 24(10): 1998-2008; Gilson et al., Follistatin Induces Muscle Hypertrophy Through Satellite Cell Proliferation and Inhibition of Both Myostatin and Activin, (2009), J. Physiol. Endocrinol., 297(1):E157-E164). Without wishing to be held to a particular theory, this observed effect may be at least partially due to FS preventing activation of the Smad2/3 pathway by myostatin and activin. Activation of the Smad2/3 pathway has been shown to result in negative regulation of muscle growth (Zhu et al., Follistatin Improves Skeletal Muscle Healing After Injury and Disease Through an Interaction with Muscle Regeneration, Angiogenesis, and Fibrosis, (2011), Musculoskeletal Pathology, 179(2):915-930).

The amino acid sequences of a typical wild-type or naturally-occurring human FS315, FS303 and FS288 protein are shown in Table 3.

TABLE 3
Exemplary Human Follistatin Isoforms

| Isoform | Follistatin Isoform Sequence |
|---|---|
| FS315 with | [symbol] [symbol] [symbol] GNCW |
| signal | LRQAKNGRCQVLYKTELSKEECCSTGRLSTSWTEED |
| sequence | VNDNTLFKWMIFNGGAPNCIPCKETCENVDCGPGKK |
| | CRMNKKNKPRCVCAPDCSNITWKGPVCGLDGKTYRN |
| | ECALLKARCKEQPELEVQYQGRCKKTCRDVFCPGSS |
| | TCVVDQTNNAYCVTCNRICPEPASSEQYLCGNDGVT |
| | YSSACHLRKATCLLGRSIGLAYEGKCIKAKSCEDIQ |
| | CTGGKKCLWDFKVGRGRCSLCDELCPDSKSDEPVCA |
| | SDNATYASECAMKEAACSSGVLLEVKHSGSCNSISE |
| | DTEEEEEDEDQDYSFPISSILEW (SEQ ID NO: |
| | 1) |
| FS315 | GNCWLRQAKNGRCQVLYKTELSKEECCSTGRLSTSW |
| | TEEDVNDNTLFKWMIFNGGAPNCIPCKETCENVDCG |
| | PGKKCRMNKKNKPRCVCAPDCSNITWKGPVCGLDGK |
| | TYRNECALLKARCKEQPELEVQYQGRCKKTCRDVFC |
| | PGSSTCVVDQTNNAYCVTCNRICPEPASSEQYLCGN |
| | DGVTYSSACHLRKATCLLGRSIGLAYEGKCIKAKSC |
| | EDIQCTGGKKCLWDFKVGRGRCSLCDELCPDSKSDE |
| | PVCASDNATYASECAMKEAACSSGVLLEVKHSGSCN |
| | SISEDTEEEEEDEDQDYSFPISSILEW (SEQ ID |
| | NO: 2) |
| FS303 with | [symbol] [symbol] [symbol] GNCW |
| signal | LRQAKNGRCQVLYKTELSKEECCSTGRLSTSWTEED |
| sequence | VNDNTLFKWMIFNGGAPNCIPCKETCENVDCGPGKK |
| | CRMNKKNKPRCVCAPDCSNITWKGPVCGLDGKTYRN |
| | ECALLKARCKEQPELEVQYQGRCKKTCRDVFCPGSS |
| | TCVVDQTNNAYCVTCNRICPEPASSEQYLCGNDGVT |
| | YSSACHLRKATCLLGRSIGLAYEGKCIKAKSCEDIQ |
| | CTGGKKCLWDFKVGRGRCSLCDELCPDSKSDEPVCA |
| | SDNATYASECAMKEAACSSGVLLEVKHSGSCNSISE |
| | DTEEEEEDEDQ (SEQ ID NO: 3) |
| FS303 | GNCWLRQAKNGRCQVLYKTELSKEECCSTGRLSTSW |
| | TEEDVNDNTLFKWMIFNGGAPNCIPCKETCENVDCG |
| | PGKKCRMNKKNKPRCVCAPDCSNITWKGPVCGLDGK |
| | TYRNECALLKARCKEQPELEVQYQGRCKKTCRDVFC |
| | PGSSTCVVDQTNNAYCVTCNRICPEPASSEQYLCGN |
| | DGVTYSSACHLRKATCLLGRSIGLAYEGKCIKAKSC |
| | EDIQCTGGKKCLWDFKVGRGRCSLCDELCPDSKSDE |
| | PVCASDNATYASECAMKEAACSSGVLLEVKHSGSCN |
| | SISEDTEEEEEDEDQ (SEQ ID NO: 4) |
| FS288 with | [symbol] [symbol] [symbol] GNCW |
| signal | LRQAKNGRCQVLYKTELSKEECCSTGRLSTSWTEE |
| sequence | DVNDNTLFKWMIFNGGAPNCIPCKETCENVDCGPG |
| | KKCRMNKKNKPRCVCAPDCSNITWKGPVCGLDGKT |
| | YRNECALLKARCKEQPELEVQYQGRCKKTCRDVFC |
| | PGSSTCVVDQTNNAYCVTCNRICPEPASSEQYLCG |
| | NDGVTYSSACHLRKATCLLGRSIGLAYEGKCIKAK |
| | SCEDIQCTGGKKCLWDFKVGRGRCSLCDELCPDSK |
| | SDEPVCASDNATYASECAMKEAACSSGVLLEVKHS |
| | GSCN (SEQ ID NO: 119) |
| FS288 | GNCWLRQAKNGRCQVLYKTELSKEECCSTGRLSTS |
| | WTEEDVNDNTLFKWMIFNGGAPNCIPCKETCENVD |
| | CGPGKKCRMNKKNKPRCVCAPDCSNITWKGPVCG |
| | LDGKTYRNECALLKARCKEQPELEVQYQGRCKKTC |
| | RDVFCPGSSTCVVDQTNNAYCVTCNRICPEPASSE |
| | QYLCGNDGVTYSSACHLRKATCLLGRSIGLAYEGK |
| | CIKAKSCEDIQCTGGKKCLWDFKVGRGRCSLCDEL |
| | CPDSKSDEPVCASDNATYASECAMKEAACSSGVLL |
| | EVKHSGSCN (SEQ ID NO: 5) |

Thus, in some embodiments, a recombinant follistatin protein suitable for the present invention is human FS315 (SEQ ID NO:1 or SEQ ID NO:2). As disclosed herein, SEQ ID NO:2 represents the canonical amino acid sequence for the human follistatin protein. In some embodiments, a follistatin protein may be a splice isoform or proteolytic variant such as FS303 (SEQ ID NO:3 or SEQ ID NO:4). In some embodiments, a follistatin protein may be a splice isoform such as FS288 (SEQ ID NO:5). In some embodiments, a suitable recombinant follistatin protein may be a homologue or an analogue of a wild-type or naturally-occurring protein. For example, a homologue or an analogue of human wild-type or naturally-occurring follistatin protein may contain one or more amino acid or domain substitutions, deletions, and/or insertions as compared to a wild-type or naturally-occurring follistatin protein (e.g., SEQ ID NO:1, SEQ ID NO:2, SEQ ID NO:3, SEQ ID NO:4, SEQ ID NO:5), while retaining substantial follistatin protein activity (e.g., myostatin or activin inhibition). Thus, in some embodiments, a recombinant follistatin protein suitable for the present invention is substantially homologous to human FS315 follistatin protein (SEQ ID NO:1). In some embodiments, a recombinant follistatin protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:1. In some embodiments, a recombinant follistatin protein suitable for the present invention is substantially identical to human FS315 follistatin protein (SEQ ID NO:1). In some embodiments, a recombinant follistatin protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:1.

In some embodiments, a recombinant follistatin protein suitable for the present invention is substantially homologous to human FS315 follistatin protein (SEQ ID NO:2). In some embodiments, a recombinant follistatin protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:2. In some embodiments, a recombinant follistatin protein suitable for the present invention is substantially identical to human FS315 follistatin protein (SEQ ID NO:2). In some embodiments, a recombinant follistatin protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:2.

In some embodiments, a recombinant follistatin protein suitable for the present invention is substantially homologous to human FS303 follistatin protein (SEQ ID NO:3). In some embodiments, a recombinant follistatin protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:3. In some embodiments, a recombinant follistatin protein suitable for the present invention is substantially identical to human FS303 follistatin protein (SEQ ID NO:3). In some embodiments, a recombinant follistatin protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:3.

In some embodiments, a recombinant follistatin protein suitable for the present invention is substantially homologous to human FS303 follistatin protein (SEQ ID NO:4). In some embodiments, a recombinant follistatin protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:4. In some embodiments, a recombinant follistatin protein suitable for the present invention is substantially identical to human FS303 follistatin protein (SEQ ID NO:4). In some embodiments, a recombinant follistatin protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID N0:4.

Thus, in some embodiments, a recombinant follistatin protein suitable for the present invention is substantially homologous to human FS288 follistatin protein (SEQ ID NO:5). In some embodiments, a recombinant follistatin protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:5. In some embodiments, a recombinant follistatin protein suitable for the present invention is substantially identical to human FS288 follistatin protein (SEQ ID NO:5). In some embodiments, a recombinant follistatin protein suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:5.

Homologues or analogues of human follistatin proteins can be prepared according to methods for altering polypeptide sequence known to one of ordinary skill in the art such as are found in references that compile such methods. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chain substitutions of one amino acid for another of the same type may often be considered a “homologous” substitution. In some embodiments, conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In some embodiments, a “conservative amino acid substitution” refers to an amino acid substitution that does not alter the relative charge or size characteristics of the protein in which the amino acid substitution is made.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology.

In some embodiments, a recombinant follistatin protein suitable for the present invention contains one or more amino acid deletions, insertions or substitutions as compared to a wild-type human follistatin protein. For example, a suitable recombinant follistatin protein may contain amino acid deletions, insertions and/or substitutions as provided in Table 4. The exemplary amino acid deletions, insertions and/or substitutions are exemplified in FS315 corresponding to SEQ ID NO:2. In some embodiments the same deletions, insertions or substitutions may be present, at the corresponding locations, in FS315 comprising the signal sequence (e.g., SEQ ID NO:1), FS303 (e.g., SEQ ID NO:3, SEQ ID NO:4) or FS288 (e.g., SEQ ID NO:5).

TABLE 4
Exemplary Recombinant Follistatin Proteins

| Sequence ID No. | |
|---|---|
| (description of | |
| mutation*) | Exemplary Recombinant Follistatin Proteins |
| SEQ ID NO: 12 | GNCWLRQAKNGRCQVLYKTELSKEECCSTGRLSTSWTEEDVNDNTLFKWMIFNGGAPNCIP |
| (deletion of amino | CKETCENVDCGPGCVCAPDCSNITWKGPVCGLDGKTYRNECALLKARCKEQPELEVQYQGR |
| acids 75 to 86; | \hat{ }\hat{ } |
| breakpoint indicated | CKKTCRDVFCPGSSTCVVDQTNNAYCVTCNRICPEPASSEQYLCGNDGVTYSSACHLRKAT |
| by \hat{ }\hat{ }) | CLLGRSIGLAYEGKCIKAKSCEDIQCTGGKKCLWDFKVGRGRCSLCDELCPDSKSDEPVCA |
| | SDNATYASECAMKEAACSSGVLLEVKHSGSCNSISEDTEEEEEDEDQDYSFPISSILEW |
| SEQ ID NO: 13 | GNCWLRQAKNGRCQVLYKTELSKEECCSTGRLSTSWTEEDVNDNTLFKWMIFNGGAPNCIP |
| (deletion of amino | CKETCENVDCGPGQSCVVDQTGSPRCVCAPDCSNITWKGPVCGLDGKTYRNECALLKARCK |
| acids 75 to 84 and | EQPELEVQYQGRCKKTCRDVFCPGSSTCVVDQTNNAYCVTCNRICPEPASSEQYLCGNDGV |
| insertion of | TYSSACHLRKATCLLGRSIGLAYEGKCIKAKSCEDIQCTGGKKCLWDFKVGRGRCSLCDEL |
| QSCVVDQTGS (SEQ | CPDSKSDEPVCASDNATYASECAMKEAACSSGVLLEVKHSGSCNSISEDTEEEEEDEDQDY |
| ID NO: 14)** | SFPISSILEW |
| SEQ ID NO: 15 | GNCWLRQAKNGRCQVLYKTELSKEECCSTGRLSTSWTEEDVNDNTLFKWMIFNGGAPNCIP |
| (K(81,82)A) | CKETCENVDCGPGKKCRMNAANKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 16 | GNCWLRQAKNGRCQVLYKTELSKEECCSTGRLSTSWTEEDVNDNTLFKWMIFNGGAPNCIP |
| (K(76,81,82)A) | CKETCENVDCGPGKACRMNAANKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 17 | GNCWLRQAKNGRCQVLYKTELSKEECCSTGRLSTSWTEEDVNDNTLFKWMIFNGGAPNCIP |
| (K82E) | CKETCENVDCGPGKKCRMNKENKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 18 | GNCWLRQAKNGRCQVLYKTELSKEECCSTGRLSTSWTEEDVNDNTLFKWMIFNGGAPNCIP |
| (K(75,76)E) | CKETCENVDCGPGEECRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 19 | GNCWLRQAKNGRCQVLYKTELSKEECCSTGRLSTSWTEEDVNDNTLFKWMIFNGGAPNCIP |
| (K(76,82)E) | CKETCENVDCGPGKECRMNKENKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 20 | GNCWLRQAKNGRCQVLYKTELSKEECCSTGRLSTSWTEEDVNDNTLFKWMIFNGGAPNCIP |
| (K(81,82)E) | CKETCENVDCGPGKKCRMNEENKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 21 | GNCWLRQAKNGRCQVLYKTELSKEECCSTGRLSTSWTEEDVNDNTLFKWMIFNGGAPNCIP |
| (K(76,81,82)E) | CKETCENVDCGPGKECRMNEENKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 22 | GNCWLRQAKNGRCQVLYKTELSKEECCSTGRLSTSWTEEDVNDNTLFKWMIFNGGAPNCIP |
| (K(76,81,82)E/ | CKETCENVDCGPGKECRMNEENKPRCECAPDCSNITWKGPVCGLDGKTYRNECALLKARCK |
| V88E) | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 23 | GNCWLRQAKNGRCQVLYKTELSKEECCSTGRLSTSWTEEDVNDNTLFKWMIFNGGAPNCIP |
| (K84E) | CKETCENVDCGPGKKCRMNKKNEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 24 | GNCWLRQAKNGRCQVLYKTELSKEECCSTGRLSTSWTEEDVNDNTLFKWMIFNGGAPNCIP |
| (K(76,84)E) | CKETCENVDCGPGKECRMNKKNEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 25 | GNCWLRQAKNGRCQVLYKTELSKEECCSTGRLSTSWTEEDVNDNTLFKWMIFNGGAPNCIP |
| (K(82,84)E) | CKETCENVDCGPGKKCRMNKENEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 26 | GNCWLRQAKNGRCQVLYKTELSKEECCSTGRLSTSWTEEDVNDNTLFKWMIFNGGAPNCIP |
| (R78E/K84E) | CKETCENVDCGPGKKCEMNKKNEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 27 | GNCWLRQAKNGRCQVLYKTELSKEECCSTGRLSTSWTEEDVNDNTLFKWMIFNGGAPNCIP |
| (K(76,82,84)E) | CKETCENVDCGPGKECRMNKENEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 28 | GNCWLRQAKNGRCQVLYKTELSKEECCSTGRLSTSWTEEDVNDNTLFKWMIFNGGAPNCIP |
| (R78E/K82E) | CKETCENVDCGPGKKCEMNKENKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 29 | GNCWLRQAKNGRCQVLYKTELSKEECCSTGRLSTSWTEEDVNDNTLFKWMIFNGGAPNCIP |
| (R78E/K(82,84)E) | CKETCENVDCGPGKKCEMNKENEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 30 | GNCWLRQAKNGRCQVLYKTELSKEECCSTGRLSTSWTEEDVNDNTLFKWMIFNGGAPNCIP |
| K(76,81)E) | CKETCENVDCGPGKECRMNEKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 31 | GNCWLRQAKNGRCQVLYKTELSKEECCSTGRLSTSWTEEDVNDNTLFKWMIFNGGAPNCIP |
| (K82T)# | CKETCENVDCGPGKKCRMNKTNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 32 | GNCWLRQAKNGRCQVLYKTELSKEECCSTGRLSTSWTEEDVNDNTLFKWMIFNGGAPNCIP |
| (P85T)# | CKETCENVDCGPGKKCRMNKKNKTRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 33 | GNCWLRQAKNGRCQVLYKTELSKEECCSTGRLSTSWTEEDVNDNTLFKWMIFNGGAPNCIP |
| (R78N/N80T)# | CKETCENVDCGPGKKCNMTKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 34 | GNCWLRQAKNGRCQVLYKTELSKEECCSTGRLSTSWTEEDVNDNTLFKWMIFNGGAPNCIP |
| (R86N/V88T)# | CKETCENVDCGPGKKCRMNKKNKPNCTCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 35 | GNCWLRQAKNGRCQVLYKTELSKEECCSTGRLSTSWTEEDVNDNTLFKWMIFNGGAPNCIP |
| (K75N/C77T/K82T)# | CKETCENVDCGPGNKTRMNKTNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 36 | GNCWLRQAKNGRCQVLYKTELSKEECCSTGRLSTSWTEEDVNDNTLFKWMIFNGGAPNCIP |
| (G74N/K76S)# | CKETCENVDCGPNKSCRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 37 | GNCWLRQAKNGRCQVLYKTELSKEECCSTGRLSTSWTEEDVNDNTLFKWMIFNGGAPNCIP |
| (G74N/K76T)# | CKETCENVDCGPNKTCRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 38 | GNCWLRQAKNGRCQVLYKTELSKEECCSTGRLSTSWTEEDVNDNTLFKWMIFNGGAPNCIP |
| (G74N/K76T/P85T)# | CKETCENVDCGPNKTCRMNKKNKTRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 39 | GNCWLRQAKNGRCQVLYKTELSKEECCSTGRLSTSWTEEDVNDNTLFKWMIFNGGAPNCIP |
| (C66S/K75N/C77T)# | CKETSENVDCGPGNKTRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 40 | GNCWLRQAKNGRCQVLYKTELSKEECCSTGRLSTSWTEEDVNDNTLFKWMIFNGGAPNCIP |
| (C66A/K75N/C77T)# | CKETAENVDCGPGNKTRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 101 | GNCWLRQAKNGRCQVLYKTELSKEECCSTGRLSTSWTEEDVNDNTLFKWMIFNGGAPNCIP |
| (K75N/C77S/K82T)# | CKETCENVDCGPGNKSRMNKTNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 102 | GNCWLRQAKNGRCQVLYKTELSKEECCSTGRLSTSWTEEDVNDNTLFKWMIFNGGAPNCIP |
| (C66S/K75N/C77S)# | CKETSENVDCGPGNKSRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 103 | GNCWLRQAKNGRCQVLYKTELSKEECCSTGRLSTSWTEEDVNDNTLFKWMIFNGGAPNCIP |
| (C66A/K75N/C77S)# | CKETAENVDCGPGNKSRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 104 | GNCWLRQAKNGRCQVLYKTELSKEECCSTGRLSTSWTEEDVNDNTLFKWMIFNGGAPNCIP |
| K(81,82)D | CKETCENVDCGPGKKCRMNDDNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 105 | GNCWLRQAKNGRCQVLYKTELSKEECCSTGRLSTSWTEEDVNDNTLFKWMIFNGGAPNCIP |
| K(76,81,82)D | CKETCENVDCGPGKDCRMNDDNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |
| SEQ ID NO: 106 | GNCWLRQAKNGRCQVLYKTELSKEECCSTGRLSTSWTEEDVNDNTLFKWMIFNGGAPNCIP |
| K(76,82)D | CKETCENVDCGPGKDCRMNKDNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW |

*numbering of amino acids corresponds to the FS315 sequence (e.g., SEQ ID NO: 2); amino acid changes as compared to the wild type FS315 sequence are underlined.
**Replacement of QSCVVDQTGS was published in J Pharmacol Exp Ther (2015) 354(2): 238. This was used as an experimental control.
#hyperglycosylation variant

In some embodiments, a recombinant follistatin protein suitable for the present invention includes hyperglycosylation mutants of the HBS region having an N-X-T/S consensus sequence. N-X-T/S consensus is a glycosylation consensus sequence motif, where X can be any amino acid except proline between Asn (N) and Thr (T) or Asn (N) and Ser (S). In some embodiments, addition of glycosylation consensus sequence masks, impairs or prevents heparin binding. In some embodiments, a recombinant follistatin protein suitable for the present invention comprises the amino acids sequences provided in Table 5 corresponding to positions 66 to 88 of the wild-type human follistatin proteins FS315, FS303 and FS288 (e.g., SEQ ID NO:2, SEQ ID NO:4 or SEQ ID NO:5). In some embodiments, hyperglycosylation variants have improved PK parameters. In some embodiments, hyperglycosylation variants do not have a net change in charge as indicated by pI (isoelectric point).

In some embodiments, deletion, insertion or substitution of amino acids within the follistatin polypeptide are within the HBS. In some embodiments, deletion, insertion or substitution of amino acids is near, or adjacent to the HBS, such as within 20, 19, 18, 17, 16, 15, 14, 13, 12, 11, 10, 9, 8, 7, 6, 5, 4, 3, 2 or 1 amino acid of the N-terminal or C-terminal amino acid of the HBS. Without wishing to be bound by theory, it is contemplated that changes within, near or adjacent to the HBS reduce heparin binding. Reduced heparin binding is contemplated to improve pharmacokinetic parameters of the recombinant protein, such as, e.g., in vivo serum half-life. Without wishing to be bound by theory, it is also contemplated that changes within, near or adjacent to the HBS may reduce immunogenicity and/or increase expression of the recombinant protein. In some embodiments, increased expression of recombinant follistatin is present with one or more of K75D, K75E, K76D, K76E, K81D, K81E, K81D, or K82E HBS mutations. In some embodiments, increased expression of recombinant follistatin is present with K82E HBS mutation. In some embodiments, substituting at least one amino acid residue (e.g. 1, 2, 3, 4, 5, 6, 7, 8, 9, or 10) within the HBS with at least one amino acid residue having a less positive charge can reduce heparin binding by the recombinant follistatin protein.

In some embodiments, amino acid substitutions within the follistatin polypeptide introduce consensus glycosylation sites within the heparin binding region (e.g., K82T, P85T, R78N/N80T, R86N/V88T, K75N/C77T/K82T, G74N/K76S, G74N/K76T, G74N/K76T/P85T, C66S/K75N/C77T, C66A/K75N/C77T K75N/C77S/K82T, C66S/K75N/C77S, C66A/K75N/C775). Subsequent glycosylation of the amino acid(s) is anticipated to mask the heparin binding domain and thus reduce binding of the recombinant protein to heparin. The presence of the glycan is also expected to mask the substituted amino acid(s) thereby modulating any potential increase in immunogencity conferred by the recombinant protein. Hyperglycosylation is also anticipated to improve the solubility and/or half-life of the recombinant protein. Exemplary hyperglycosylation variants are shown, as indicated, in Tables 4, 5 and 9.

TABLE 5
Exemplary FS Sequences

| | FS sequence correspond- |
|---|---|
| | ing to amino acids 66 |
| Sequence ID No. | to 88 of wild-type |
| (description of mutation*) | follistatin* |
| FS-WT amino acids 66 to | CENVDCGPGKKCRMNKKNKPRCV |
| 88 SEQ ID NO: 107 | |
| FSdelHBS (FSD2) (FS315; | CENVDCGPGSTCVVDQTNNAYCV |
| FS303; FS288) | |
| SEQ ID NO: 108 | |
| FS315HBS (del75-86) | CENVDCGPG------------CV |
| SEQ ID NO: 109 | |
| SEQ ID NO: 41 | CENVDCGPGQSCVVDQTGSPRCV |
| FS315delHBS/FSTL-D2 | |
| (deletion of amino acids | |
| 75 to 84 and insertion | |
| of QSCVVDQTGS (SEQ ID NO: | |
| 14))** | |
| SEQ ID NO: 42 | CENVDCGPGKKCRMNAANKPRCV |
| (K(81,82)A) | |
| SEQ ID NO: 43 | CENVDCGPGKACRMNAANKPRCV |
| (K(76,81,82)A) | |
| SEQ ID NO: 44 | CENVDCGPGKKCRMNKENKPRCV |
| (K82E) | |
| SEQ ID NO: 45 | CENVDCGPGEECRMNKKNKPRCV |
| (K(75,76)E) | |
| SEQ ID NO: 46 | CENVDCGPGKECRMNKENKPRCV |
| (K(76,82)E) | |
| SEQ ID NO: 47 | CENVDCGPGKKCRMNEENKPRCV |
| (K(81,82)E) | |
| SEQ ID NO: 48 | CENVDCGPGKECRMNEENKPRCV |
| (K(76,81,82)E) | |
| SEQ ID NO: 49 | CENVDCGPGKECRMNEENKPRCE |
| (K(76,81,82)E/V88E) | |
| SEQ ID NO: 50 | CENVDCGPGKKCRMNKKNEPRCV |
| (K84E) | |
| SEQ ID NO: 51 | CENVDCGPGKECRMNKKNEPRCV |
| (K(76,84)E) | |
| SEQ ID NO: 52 | CENVDCGPGKKCRMNKENEPRCV |
| (K(82,84)E) | |
| SEQ ID NO: 53 | CENVDCGPGKKCEMNKKNEPRCV |
| (R78E/K84E) | |
| SEQ ID NO: 54 | CENVDCGPGKECRMNKENEPRCV |
| (K(76,82,84)E) | |
| SEQ ID NO: 55 | CENVDCGPGKKCEMNKENKPRCV |
| (R78E/K82E) | |
| SEQ ID NO: 56 | CENVDCGPGKKCEMNKENEPRCV |
| (R78E/K(82,84)E) | |
| SEQ ID NO: 57 | CENVDCGPGKECRMNEKNKPRCV |
| (K(76,81)E) | |
| SEQ ID NO: 58 | CENVDCGPGKKCRMNKTNKPRCV |
| (K82T)# | |
| SEQ ID NO: 59 | CENVDCGPGKKCRMNKKNKTRCV |
| (P85T)# | |
| SEQ ID NO: 60 | CENVDCGPGKKCNMTKKNKPRCV |
| (R78N/N80T)# | |
| SEQ ID NO: 61 | CENVDCGPGKKCRMNKKNKPNCT |
| (R86N/V88T)# | |
| SEQ ID NO: 62 | CENVDCGPGNKTRMNKTNKPRCV |
| (K75N/C77T/K82T)# | |
| SEQ ID NO: 63 | CENVDCGPNKSCRMNKKNKPRCV |
| (G74N/K76S)# | |
| SEQ ID NO: 64 | CENVDCGPNKTCRMNKKNKPRCV |
| (G74N/K76T)# | |
| SEQ ID NO: 65 | CENVDCGPNKTCRMNKKNKTRCV |
| (G74N/K76T/P85T)# | |
| SEQ ID NO: 66 | SENVDCGPGNKTRMNKKNKPRCV |
| (C66S/K75N/C77T)# | |
| SEQ ID NO: 67 | AENVDCGPGNKTRMNKKNKPRCV |
| (C66A/K75N/C77T)# | |
| SEQ ID NO: 111 | CENVDCGPGNKSRMNKTNKPRCV |
| (K75N/C77S/K82T)# | |
| SEQ ID NO: 112 | SENVDCGPGNKSRMNKKNKPRCV |
| (C66S/K75N/C77S)# | |
| SEQ ID NO: 113 | AENVDCGPGNKSRMNKKNKPRCV |
| (C66A/K75N/C77S)# | |
| SEQ ID NO: 114 | CENVDCGPGKKCRMNDDNKPRCV |
| K(81,82)D | |
| SEQ ID NO: 115 | CENVDCGPGKDCRMNDDNKPRCV |
| K(76,81,82)D | |
| SEQ ID NO: 116 | CENVDCGPGKDCRMNKDNKPRCV |
| K(76,82)D | |

*numbering of amino acids corresponds to the FS315 sequence (e.g., SEQ ID NO: 2); amino acid changes are underlines.
**Replacement of QSCVVDQTGS was published in J Pharmacol Exp Ther (2015) 354(2): 238. This was used as an experimental control.
#hyperglycoslyation variant.

Follistatin Fusion Proteins

It is contemplated that a suitable recombinant follistatin protein can be in a fusion protein configuration. For example, a recombinant follistatin protein suitable for the present invention may be a fusion protein between a follistatin domain and another domain or moiety that typically can facilitate a therapeutic effect of follistatin by, for example, enhancing or increasing stability, potency and/or delivery of follistatin protein, or reducing or eliminating immunogenicity, or clearance. Such suitable domains or moieties for a follistatin fusion protein include but are not limited to Fc domain, XTEN domain, or human albumin fusions.

Fc Domain

In some embodiments, a suitable recombinant follistatin protein contains an Fc domain or a portion thereof that binds to the FcRn receptor. As a non-limiting example, a suitable Fc domain may be derived from an immunoglobulin subclass such as IgG. In some embodiments, a suitable Fc domain is derived from IgG1, IgG2, IgG3, or IgG4. In some embodiments, a suitable Fc domain is derived from IgM, IgA, IgD, or IgE. Particularly suitable Fc domains include those derived from human or humanized antibodies. In some embodiments, a suitable Fc domain is a modified Fc portion, such as a modified human Fc portion.

In some embodiments, a suitable Fc domain comprises an amino acid sequence as provided in Table 6.

TABLE 6
Exemplary Fc domains

| Sequence | |
|---|---|
| ID No. | |
| (description) | Fc Domain* |
| SEQ ID NO: 6 | DKTHTCPPCPAPELLGGPSVFLFPPKPKDTLMIS |
| (wild-type | RTPEVTCVVVDVSHEDPEVKFNWYVDGVEVHNAK |
| human IgG1 Fc) | TKPREEQYNSTYRVVSVLTVLHQDWLNGKEYKCK |
| | VSNKALPAPIEKTISKAKGQPREPQVYTLPPSRD |
| | ELTKNQVSLTCLVKGFYPSDIAVEWESNGQPENN |
| | YKTTPPVLDSDGSFFLYSKLTVDKSRWQQGNVFS |
| | CSVMHEALHNHYTQKSLSLSPGK |
| SEQ ID NO: 7 | DKTHTCPPCPAPEAAGGPSVFLFPPKPKDTLMIS |
| (human IgG1 | RTPEVTCVVVDVSHEDPEVKFNWYVDGVEVHNAK |
| Fc-LALA) | TKPREEQYNSTYRVVSVLTVLHQDWLNGKEYKCK |
| | VSNKALPAPIEKTISKAKGQPREPQVYTLPPSRD |
| | ELTKNQVSLTCLVKGFYPSDIAVEWESNGQPENN |
| | YKTTPPVLDSDGSFFLYSKLTVDKSRWQQGNVFS |
| | CSVMHEALHNHYTQKSLSLSPGK |
| SEQ ID NO: 8 | DKTHTCPPCPAPELLGGPSVFLFPPKPKDTLMIS |
| (human IgG1 | RTPEVTCVVVDVSHEDPEVKFNWYVDGVEVHNAK |
| Fc-NHance) | TKPREEQYNSTYRVVSVLTVLHQDWLNGKEYKCK |
| | VSNKALPAPIEKTISKAKGQPREPQVYTLPPSRD |
| | ELTKNQVSLTCLVKGFYPSDIAVEWESNGQPENN |
| | YKTTPPVLDSDGSFFLYSKLTVDKSRWQQGNVFS |
| | CSVMHEALKFHYTQKSLSLSPGK |
| SEQ ID NO: 9 | DKTHTCPPCPAPEAAGGPSVFLFPPKPKDTLMIS |
| (human IgG1 | RTPEVTCVVVDVSHEDPEVKFNWYVDGVEVHNAK |
| Fc-LALA + | TKPREEQYNSTYRVVSVLTVLHQDWLNGKEYKCK |
| NHance) | VSNKALPAPIEKTISKAKGQPREPQVYTLPPSRD |
| | ELTKNQVSLTCLVKGFYPSDIAVEWESNGQPENN |
| | YKTTPPVLDSDGSFFLYSKLTVDKSRWQQGNVFS |
| | CSVMHEALKFHYTQKSLSLSPGK |
| SEQ ID NO: 10 | EPKSCDKTHTCPPCPAPELLGGPSVFLFPPKPKD |
| | TLMISRTPEVTCVVVDVSHEDPEVKFNWYVDGVE |
| | VHNAKTKPREEQYNSTYRVVSVLTVLHQDWLNGK |
| | EYKCKVSNKALPAPIEKTISKAKGQPREPQVYTL |
| | PPSRDELTKNQVSLTCLVKGFYPSDIAVEWESNG |
| | QPENNYKTTPPVLDSDGSFFLYSKLTVDKSRWQQ |
| | GNVFSCSVMHEALHNHYTQKSLSLSPGK |
| SEQ ID NO: 11 | KTHTCPPCPAPELLGGPSVFLFPPKPKDTLMISR |
| | TPEVTCVVVDVSHEDPEVKFNWYVDGVEVHNAKT |
| | KPREEQYNSTYRVVSVLTVLHQDWLNGKEYKCKV |
| | SNKALPAPIEKTISKAKGQPREPQVYTLPPSRDE |
| | LTKNQVSLTCLVKGFYPSDIAVEWESNGQPENNY |
| | KTTPPVLDSDGSFFLYSKLTVDKSRWQQGNVFSC |
| | SVMHEALHNHYTQKSLSLSPGK |

*numbering of amino acids based on EU numbering. LALA and NHance mutations are underlined.

In some embodiments, a suitable Fc domain comprises an amino acid sequence at least 50%, 55%, 60%, 65%, 70%, 75%, 80%, 85%, 90%, 91%, 92%, 93%, 94%, 95%, 96%, 97%, 98%, 99% or more homologous or identical to SEQ ID NO:6, SEQ ID NO:7, SEQ ID NO:8, SEQ ID NO:9, SEQ ID NO:10 or SEQ ID NO:11.

It is contemplated that improved binding between the Fc domain and the FcRn receptor results in prolonged serum half-life of the recombinant protein. Thus, in some embodiments, a suitable Fc domain comprises one or more amino acid mutations that lead to improved binding to FcRn. Various mutations within the Fc domain that effect improved binding to FcRn are known in the art and can be adapted to practice the present invention. In some embodiments, a suitable Fc domain comprises one or more mutations at one or more positions corresponding to Thr 250, Met 252, Ser 254, Thr 256, Thr 307, Glu 380, Met 428, His 433 and/or Asn 434 of human IgG1, according to EU numbering.

In some embodiments, a suitable Fc domain comprises one or more mutations at one or more positions corresponding to L234, L235, H433 and N434 of human IgG1, according to EU numbering.

The Fc portion of a recombinant fusion protein may lead to targeting of cells that express Fc receptors leading to pro-inflammatory effects. Some mutations in the Fc domain reduce binding of the recombinant protein to the Fc gamma receptor and thereby inhibit effector functions. In one embodiment, effector function is antibody-dependent cell-mediated cytotoxicity (ADCC). For example, a suitable Fc domain may contain mutations of L234A (Leu234A1a) and/or L235A (Leu235A1a) (EU numbering). In some embodiments the L234A and L235A mutations are also referred to as the LALA mutations. As a non-limiting example, a suitable Fc domain may contain mutations L234A and L235A (EU numbering). An exemplary Fc domain sequence comprising the L234A and L235A mutations is shown as SEQ ID NO:7 in Table 6.

In some embodiments, a suitable Fc domain may contain mutations of H433K (His433Lys) and/or N434F (Asn434Phe) (EU numbering). As a non-limiting example, a suitable Fc domain may contain mutations H433K and N434F (EU numbering). In some embodiments the H433K and N434F mutations are also referred to as the NHance mutations. An exemplary Fc domain sequence incorporating the mutations H433K and N434F is shown as SEQ ID NO:8 in Table 6.

In some embodiments, a suitable Fc domain may contain mutations of L234A (Leu234A1a), L235A (Leu235A1a), H433K (His433Lys) and/or N434F (Asn434Phe) (EU numbering). As a non-limiting example, a suitable Fc domain may contain mutations L234A, L235A, H433K and N434F (EU numbering). An exemplary Fc domain sequence incorporating the mutations L234A, L235A, H433K and N434F is shown as SEQ ID NO:9 in Table 6.

Additional amino acid substitutions that can be included in the Fc domain include those described in, e.g., U.S. Pat. Nos. 6,277,375; 8,012,476; and 8,163,881, which are incorporated herein by reference.

Linker or Spacer

A follistatin domain may be directly or indirectly linked to an Fc domain. In some embodiments, a suitable recombinant follistatin protein contains a linker or spacer that joins a follistatin domain and an Fc domain. An amino acid linker or spacer is generally designed to be flexible or to interpose a structure, such as an alpha-helix, between the two protein moieties. A linker or spacer can be relatively short, or can be longer. Typically, a linker or spacer contains for example 3-100 (e.g., 5-100, 10-100, 20-100 30-100, 40-100, 50-100, 60-100, 70-100, 80-100, 90-100, 5-55, 10-50, 10-45, 10-40, 10-35, 10-30, 10-25, 10-20) amino acids in length. In some embodiments, a linker or spacer is equal to or longer than 2, 3, 4, 5, 6, 7, 8, 9, 10, 15, 20, 25, 30, 35, 40, 45, 50, 55, 60, 65, 70, 75, 80, 85, 90, 95, or 100 amino acids in length. Typically, a longer linker may decrease steric hindrance. In some embodiments, a linker will comprise a mixture of glycine and serine residues. In some embodiments, the linker may additionally comprise threonine, proline and/or alanine residues. Thus, in some embodiments, the linker comprises between 10-100, 10-90, 10-80, 10-70, 10-60, 10-50, 10-40, 10-30, 10-20, 10-15 amino acids. In some embodiments, the linker comprises at least 10, 15, 20, 25, 30, 35, 40, 45, 50, 55, 60, 65, 70, 75, 80, 85, 90, or 95 amino acids. In some embodiments, the linker is not a linker consisting of ALEVLFQGP (SEQ ID NO:68).

As non-limiting examples, linkers or spacers suitable for the present invention include but are not limited to:

(SEQ ID NO: 69)
GGG;
(GAG linker, SEQ ID NO: 70)
GAPGGGGGAAAAAGGGGGGAP;
(GAG2 linker, SEQ ID NO: 71)
GAPGGGGGAAAAAGGGGGGAPGGGGGAAAAAGGGGGGAP;
and
(GAG3 linker, SEQ ID NO: 72)
GAPGGGGGAAAAAGGGGGGAPGGGGGAAAAAGGGGGGAPGGGGGAAAAAGG
GGGGAP.

Suitable linkers or spacers also include those having an amino acid sequence at least 50%, 55%, 60%, 65%, 70%, 75%, 80%, 85%, 90%, 91%, 92%, 93%, 94%, 95%, 96%, 97%, 98%, 99% or more homologous or identical to the above exemplary linkers, e.g., GAG linker (SEQ ID NO:70), GAG2 linker (SEQ ID NO:71), or GAG3 linker (SEQ ID NO:72). Additional linkers suitable for use with some embodiments may be found in US20120232021, filed on Mar. 2, 2012, the disclosure of which is hereby incorporated by reference in its entirety.

In some embodiments, a linker is provided that associates the follistatin polypeptide with the Fc domain without substantially affecting the ability of the follistatin polypeptide to bind to any of its cognate ligands (e.g., activin A, myostatin, heparin, etc.). In some embodiments, a linker is provided such that the binding of a follistatin peptide to heparin is not altered as compared to the follistatin polypeptide alone.

Exemplary Follistatin Fusion Proteins

In particular embodiments, a suitable recombinant follistatin fusion protein includes a follistatin polypeptide and an Fc domain, wherein the follistatin polypeptide comprises an amino acid sequence at least 50%, 55%, 60%, 65%, 70%, 75%, 80%, 85%, 90%, 91%, 92%, 93%, 94%, 95%, 96%, 97%, 98%, 99% or 100% identical to the wild-type human FS315 protein (SEQ ID NO:1 or SEQ ID NO:2), FS303 protein (SEQ ID NO:3 or SEQ ID NO:4) or FS288 (SEQ ID NO:5). In particular embodiments, a suitable recombinant follistatin fusion protein includes a follistatin polypeptide, an Fc domain, and a linker that associates the follistatin polypeptide with the Fc domain, wherein the follistatin polypeptide comprises an amino acid sequence at least 50%, 55%, 60%, 65%, 70%, 75%, 80%, 85%, 90%, 91%, 92%, 93%, 94%, 95%, 96%, 97%, 98%, 99% or 100% identical to the wild-type human FS315 protein (SEQ ID NO:1) or FS315 protein (SEQ ID NO:2). Typically, a suitable recombinant follistatin fusion protein is capable of binding to activin A and myostatin. In some embodiments, a suitable recombinant follistatin fusion protein has an in vivo half-life ranging from about 0.5-6 days (e.g., about 0.5-5.5 days, about 0.5-5 days, about 1-5 days, about 1.5-5 days, about 1.5-4.5 days, about 1.5-4.0 days, about 1.5-3.5 days, about 1.5-3 days, about 1.5-2.5 days, about 2-6 days, about 2-5.5 days, about 2-5 days, about 2-4.5 days, about 2-4 days, about 2-3.5 days, about 2-3 days). In some embodiments, a suitable recombinant follistatin fusion protein has an in vivo half-life ranging from about 2-10 days (e.g., ranging from about 2.5-10 days, from about 3-10 days, from about 3.5-10 days, from about 4-10 days, from about 4.5-10 days, from about 5-10 days, from about 3-8 days, from about 3.5-8 days, from about 4-8 days, from about 4.5-8 days, from about 5-8 days, from about 3-6 days, from about 3.5-6 days, from about 4-6 days, from about 4.5-6 days, from about 5-6 days).

As non-limiting examples, suitable follistatin Fc fusion proteins may have an amino acid sequence shown in Table 7.

TABLE 7
Exemplary Follistatin Fc Fusion Proteins

| Sequence ID No. | |
|---|---|
| (description of | |
| mutation*) | Exemplary Recombinant Follistatin-Fc Fusion Proteins # |
| SEQ ID NO: 73 | GNCWLRQAKNGRCQVLYKTELSKEECCSTGRLSTSWTEEDVNDNTLFKWMIFNGGAPNCIP |
| (deletion of amino | CKETCENVDCGPGCVCAPDCSNITWKGPVCGLDGKTYRNECALLKARCKEQPELEVQYQGR |
| acids 75 to 86; | \hat{ }\hat{ } |
| breakpoint indicated | CKKTCRDVFCPGSSTCVVDQTNNAYCVTCNRICPEPASSEQYLCGNDGVTYSSACHLRKAT |
| by \hat{ }\hat{ }) | CLLGRSIGLAYEGKCIKAKSCEDIQCTGGKKCLWDFKVGRGRCSLCDELCPDSKSDEPVCA |
| | SDNATYASECAMKEAACSSGVLLEVKHSGSCNSISEDTEEEEEDEDQDYSFPISSILEW [symbol] |
| | [symbol] [symbol] |
| | [symbol] [symbol] |
| | [symbol] [symbol] |
| | [symbol] [symbol] |
| SEQ ID NO:74 | GNCWLRQAKNGRCQVLYKTELSKEECCSTGRLSTSWTEEDVNDNTLFKWMIFNGGAPNCIP |
| (deletion of amino | CKETCENVDCGPGQSCVVDQTGSPRCVCAPDCSNITWKGPVCGLDGKTYRNECALLKARCK |
| acids 75 to 84 and | EQPELEVQYQGRCKKTCRDVFCPGSSTCVVDQTNNAYCVTCNRICPEPASSEQYLCGNDGV |
| insertion of | TYSSACHLRKATCLLGRSIGLAYEGKCIKAKSCEDIQCTGGKKCLWDFKVGRGRCSLCDEL |
| QSCVVDQTGS | CPDSKSDEPVCASDNATYASECAMKEAACSSGVLLEVKHSGSCNSISEDTEEEEEDEDQDY |
| (SEQ ID NO: 14)** | SFPISSILEW [symbol] [symbol] |
| | [symbol] [symbol] |
| | [symbol] [symbol] |
| | [symbol] [symbol] |
| SEQ ID NO: 75 | GNCWLRQAKNGRCQVLYKTELSKEECCSTGRLSTSWTEEDVNDNTLFKWMIFNGGAPNCIP |
| (K(81,82)A) | CKETCENVDCGPGKKCRMNAANKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 76 | GNCWLRQAKNGRCQVLYKTELSKEECCSTGRLSTSWTEEDVNDNTLFKWMIFNGGAPNCIP |
| (K(76,81,82)A) | CKETCENVDCGPGKACRMNAANKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 77 | GNCWLRQAKNGRCQVLYKTELSKEECCSTGRLSTSWTEEDVNDNTLFKWMIFNGGAPNCIP |
| (K82E) | CKETCENVDCGPGKKCRMNKENKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 78 | GNCWLRQAKNGRCQVLYKTELSKEECCSTGRLSTSWTEEDVNDNTLFKWMIFNGGAPNCIP |
| (K(75,76)E) | CKETCENVDCGPGEECRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 79 | GNCWLRQAKNGRCQVLYKTELSKEECCSTGRLSTSWTEEDVNDNTLFKWMIFNGGAPNCIP |
| (K(76,82)E) | CKETCENVDCGPGKECRMNKENKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 80 | GNCWLRQAKNGRCQVLYKTELSKEECCSTGRLSTSWTEEDVNDNTLFKWMIFNGGAPNCIP |
| (K(81,82)E) | CKETCENVDCGPGKKCRMNEENKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 81 | GNCWLRQAKNGRCQVLYKTELSKEECCSTGRLSTSWTEEDVNDNTLFKWMIFNGGAPNCIP |
| (K(76,81,82)E) | CKETCENVDCGPGKECRMNEENKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 82 | GNCWLRQAKNGRCQVLYKTELSKEECCSTGRLSTSWTEEDVNDNTLFKWMIFNGGAPNCIP |
| (K(76,81,82)E/V88E) | CKETCENVDCGPGKECRMNEENKPRCECAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 83 | GNCWLRQAKNGRCQVLYKTELSKEECCSTGRLSTSWTEEDVNDNTLFKWMIFNGGAPNCIP |
| (K84E) | CKETCENVDCGPGKKCRMNKKNEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 84 | GNCWLRQAKNGRCQVLYKTELSKEECCSTGRLSTSWTEEDVNDNTLFKWMIFNGGAPNCIP |
| (K(76,84)E) | CKETCENVDCGPGKECRMNKKNEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 85 | GNCWLRQAKNGRCQVLYKTELSKEECCSTGRLSTSWTEEDVNDNTLFKWMIFNGGAPNCIP |
| (K(82,84)E) | CKETCENVDCGPGKKCRMNKENEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 86 | GNCWLRQAKNGRCQVLYKTELSKEECCSTGRLSTSWTEEDVNDNTLFKWMIFNGGAPNCIP |
| (R78E/K84E) | CKETCENVDCGPGKKCEMNKKNEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 87 | GNCWLRQAKNGRCQVLYKTELSKEECCSTGRLSTSWTEEDVNDNTLFKWMIFNGGAPNCIP |
| (K(76,82,84)E) | CKETCENVDCGPGKECRMNKENEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 88 | GNCWLRQAKNGRCQVLYKTELSKEECCSTGRLSTSWTEEDVNDNTLFKWMIFNGGAPNCIP |
| (R78E/K82E) | CKETCENVDCGPGKKCEMNKENKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 89 | GNCWLRQAKNGRCQVLYKTELSKEECCSTGRLSTSWTEEDVNDNTLFKWMIFNGGAPNCIP |
| (R78E/K(82,84)E) | CKETCENVDCGPGKKCEMNKENEPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 90 | GNCWLRQAKNGRCQVLYKTELSKEECCSTGRLSTSWTEEDVNDNTLFKWMIFNGGAPNCIP |
| K(76,81)E) | CKETCENVDCGPGKECRMNEKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 91 | GNCWLRQAKNGRCQVLYKTELSKEECCSTGRLSTSWTEEDVNDNTLFKWMIFNGGAPNCIP |
| (K82T) | CKETCENVDCGPGKKCRMNKTNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 92 | GNCWLRQAKNGRCQVLYKTELSKEECCSTGRLSTSWTEEDVNDNTLFKWMIFNGGAPNCIP |
| (P85T) | CKETCENVDCGPGKKCRMNKKNKTRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 93 | GNCWLRQAKNGRCQVLYKTELSKEECCSTGRLSTSWTEEDVNDNTLFKWMIFNGGAPNCIP |
| (R78N/N80T) | CKETCENVDCGPGKKCNMTKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 94 | GNCWLRQAKNGRCQVLYKTELSKEECCSTGRLSTSWTEEDVNDNTLFKWMIFNGGAPNCIP |
| (R86N/V88T) | CKETCENVDCGPGKKCRMNKKNKPNCTCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 95 | GNCWLRQAKNGRCQVLYKTELSKEECCSTGRLSTSWTEEDVNDNTLFKWMIFNGGAPNCIP |
| (K75N/C77T/K82T) | CKETCENVDCGPGNKTRMNKTNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 96 | GNCWLRQAKNGRCQVLYKTELSKEECCSTGRLSTSWTEEDVNDNTLFKWMIFNGGAPNCIP |
| (G74N/K76S) | CKETCENVDCGPNKSCRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 97 | GNCWLRQAKNGRCQVLYKTELSKEECCSTGRLSTSWTEEDVNDNTLFKWMIFNGGAPNCIP |
| (G74N/K76T) | CKETCENVDCGPNKTCRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 98 | GNCWLRQAKNGRCQVLYKTELSKEECCSTGRLSTSWTEEDVNDNTLFKWMIFNGGAPNCIP |
| (G74N/K76T/P85T) | CKETCENVDCGPNKTCRMNKKNKTRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 99 | GNCWLRQAKNGRCQVLYKTELSKEECCSTGRLSTSWTEEDVNDNTLFKWMIFNGGAPNCIP |
| (C66S/K75N/C77T) | CKETSENVDCGPGNKTRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 100 | GNCWLRQAKNGRCQVLYKTELSKEECCSTGRLSTSWTEEDVNDNTLFKWMIFNGGAPNCIP |
| (C66A/K75N/C77T) | CKETAENVDCGPGNKTRMNKKNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 117 | GNCWLRQAKNGRCQVLYKTELSKEECCSTGRLSTSWTEEDVNDNTLFKWMIFNGGAPNCIP |
| (K(76,82)D) | CKETCENVDCGPGKDCRMNKDNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 120 | GNCWLRQAKNGRCQVLYKTELSKEECCSTGRLSTSWTEEDVNDNTLFKWMIFNGGAPNCIP |
| (K(81,82)D) | CKETCENVDCGPGKKCRMNDDNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |
| SEQ ID NO: 118 | GNCWLRQAKNGRCQVLYKTELSKEECCSTGRLSTSWTEEDVNDNTLFKWMIFNGGAPNCIP |
| (K(76,81,82)D) | CKETCENVDCGPGKDCRMNDDNKPRCVCAPDCSNITWKGPVCGLDGKTYRNECALLKARCK |
| | EQPELEVQYQGRCKKTCRDVFCPGSSTCVVDQTNNAYCVTCNRICPEPASSEQYLCGNDGV |
| | TYSSACHLRKATCLLGRSIGLAYEGKCIKAKSCEDIQCTGGKKCLWDFKVGRGRCSLCDEL |
| | CPDSKSDEPVCASDNATYASECAMKEAACSSGVLLEVKHSGSCNSISEDTEEEEEDEDQDY |
| | SFPISSILEW [symbol] [symbol] |
| | [symbol] [symbol] |
| | [symbol] [symbol] |
| | [symbol] [symbol] |

*numbering of the FS amino acids corresponds to the FS315 sequence (e.g., SEQ ID NO: 2).
**Replacement of QSCVVDQTGS was published in J Pharmacol Exp Ther (2015) 354(2): 238. This was used as an experimental control.
# sequence in bold and italic corresponds to human IgG1Fc comprising LALA mutations at positions 234 and 235 (underlined and according to EU numbering) (SEQ ID NO: 7).

In some embodiments, the recombinant follistatin-Fc fusion proteins may be designated as FS315K(81,82)A-hFcLALA, FS315K(81,82)A-GGG-hFcLALA, FS315K(76,81,82)A-hFcLALA, FS303K(76,81,82)A-hFcLALA, FS315K(76,81,82)A-GGG-hFcLALA, FS303K(76,81,82)A-GGG-hFcLALA, FS315K82T-hFcLALA, FS303K82T-hFcLALA, FS315K82T-GGG-hFcLALA, FS303K82T-GGG-hFcLALA, FS315K(76,81)E-hFcLALA, FS315K(76,81,82)E/V88E-hFcLALA, FS315WT-hFcLALA, FS315K(75,76)E-hFcLALA, FS315K(76,82)E-hFcLALA, FS315K(76,82)D-hFcLALA, FS315R86N/V88T-hFcLALA, FS315K75N/C77T/K82T-hFcLALA, FS315K75N/C77S/K82T-hFcLALA, FS315 de175-86-hFcLALA, FS315K(81,82)E-hFcLALA, FS315K(81,82)D-hFcLALA FS315K82E-hFcLALA, FS315K(76,81,82)E-hFcLALA, FS315K(76,81,82)D-hFcLALA, FS315R78N/N80T-hFcLALA, FS315P85T-hFcLALA, FS315K(76,81)E-hFcLALA or FS315K75N/C77N/K82T-hFcLALA.

It is contemplated that a follistatin-Fc fusion protein may be provided in various configurations including homodimeric or monomeric configurations. For example, a suitable homodimeric configuration may be designed to have the C-terminal end of fusion partner (e.g., a follistatin polypeptide plus linker) attached to the N-terminal end of both Fc polypeptide strands. A suitable monomeric configuration may be designed to have the C-terminal end of fusion partner (e.g., a follistatin polypeptide plus linker) fused to one Fc dimer, or to one Fc monomer. A monomeric configuration may decrease steric hindrance.

As used herein, “percent (%) amino acid sequence identity” with respect to a reference protein sequence (e.g., a reference follistatin protein sequence) identified herein is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, world threshold (T)=11. HSP score (S) and HSP S2 parameters are dynamic values and are established by the program itself, depending upon the composition of the particular sequence, however, the minimum values may be adjusted and are set as indicated above.

In some embodiments, a recombinant follistatin-Fc fusion protein inhibits the binding and/or activity of myostatin. In some embodiments, a recombinant follistatin-Fc fusion protein has a K_{D} of greater than about 0.1 pM, greater than about 0.5 pM, greater than about 1 pM, greater than about 5 pM, greater than about 10 pM, greater than about 50 pM, greater than about 100 pM, greater than about 500 pM or greater than about 1000 pM when binding myostatin. The affinity of a recombinant follistatin-Fc fusion protein may be measured, for example, in a surface plasmon resonance assay, such as a BIAcore assay.

In some embodiments, a recombinant follistatin-Fc fusion protein inhibits the binding and/or activity of activin A. In some embodiments, a recombinant follistatin-Fc fusion protein has a K_{D} of greater than about 0.1 pM, greater than about 0.5 pM, greater than about 1 pM, greater than about 5 pM, greater than about 10 pM, greater than about 50 pM, greater than about 100 pM, greater than about 500 pM or greater than about 1000 pM when binding activin A. The affinity of a recombinant follistatin-Fc fusion protein may be measured, for example, in a surface plasmon resonance assay, such as a BIAcore assay.

In some embodiments, a recombinant follistatin-Fc fusion protein has a reduced binding affinity for heparin as compared to the binding affinity of a wild-type follistatin-Fc protein for heparin. In some embodiments, a recombinant follistatin-Fc fusion protein has a K_{D} of greater than about 0.01 nM, greater than about 0.05 nM, greater than about 0.1 nM, greater than about 0.5 nM, greater than about 1 nM, greater than about 5 nM, greater than about 10 nM, greater than about 50 nM, greater than about 100 nM, greater than about 150 nM, greater than about 200 nM, greater than about 250 nM or greater than about 500 nM when binding heparin.

In some embodiments, a recombinant follistatin-Fc fusion protein has a K_{D} of greater than about 1 nM, greater than about 5 nM, greater than about 10 nM, greater than about 50 nM, greater than about 100 nM, greater than about 500 nM, or greater than about 1000 nM when binding a Fc receptor. In some embodiments, the Fc receptor is an Fcγ receptor. In some embodiments, the Fcγ receptor is FcγRI, FcγRIIA, FcγRIIB, FcγRIIIA or FcγRIIIB.

In some embodiments, a recombinant follistatin-Fc fusion protein has minimal or no appreciable binding to BMP-9. In some embodiments, a recombinant follistatin-Fc fusion protein has minimal or no appreciable binding or BMP-10. In some embodiments, the minimal or no appreciable binding is determined in the range of 190 pM to 25000 pM.

In some embodiments, a recombinant follistatin-Fc fusion protein, is characterized by an IC₅₀ below about 20 nM, below about 15 nM, below about 10 nM, below about 5 nM, below about 4 nM, below about 3 nM, below about 2 nM, below about 1 nM, below about 0.5 nM, below about 0.25 nM, below about 0.1 nM, below about 0.05 nM or below about 0.01 nM in a myostatin stimulation assay.

In some embodiments, a recombinant follistatin-Fc fusion protein is characterized by an IC₅₀ below about 20 nM, below about 15 nM, below about 10 nM, below about 5 nM, below about 4 nM, below about 3 nM, below about 2 nM, below about 1 nM, below about 0.5 nM, below about 0.25 nM, below about 0.1 nM, below about 0.05 nM or below about 0.01 nM in an activin A stimulation assay.

In some embodiments, administration of a recombinant follistatin-Fc fusion protein in vivo results in an increase in the mass of a muscle relative to a control. In some embodiments, the mass of the muscle is, for example, the weight of the muscle. In some embodiments the muscle is one or more skeletal muscles, for example, those presented in Table 1. In some embodiments, the muscle selected from the group consisting of diaphragm, triceps, soleus, tibialis anterior, gastrocnemius, extensor digitorum longus, rectus abdominus, quadriceps, and combinations thereof.

### Production of Recombinant Follistatin or Recombinant Follistatin-Fc Fusion Proteins

A recombinant follistatin protein or recombinant follistatin-Fc fusion protein suitable for the present invention may be produced by any available means. For example, a recombinant follistatin protein or recombinant follistatin-Fc fusion protein may be recombinantly produced by utilizing a host cell system engineered to express a recombinant follistatin protein or recombinant follistatin-Fc fusion protein-encoding nucleic acid. Alternatively or additionally, a recombinant follistatin protein or recombinant follistatin-Fc fusion protein may be produced by activating endogenous genes. Alternatively or additionally, a recombinant follistatin protein or recombinant follistatin-Fc fusion protein may be partially or fully prepared by chemical synthesis.

Where proteins are recombinantly produced, any expression system can be used. To give but a few examples, known expression systems include, for example, E. coli, egg, baculovirus, plant, yeast, or mammalian cells, for example CHO cells and/or other mammalian cells described below.

In some embodiments, recombinant follistatin proteins or recombinant follistatin-Fc fusion proteins suitable for the present invention are produced in mammalian cells. Non-limiting examples of mammalian cells that may be used in accordance with the present invention include BALB/c mouse myeloma line (NSW, ECACC No: 85110503); human retinoblasts (PER.C6, CruCell, Leiden, The Netherlands); monkey kidney CV1 line transformed by SV40 (COS-7, ATCC CRL 1651); human embryonic kidney line (HEK293 or 293 cells subcloned for growth in suspension culture, Graham et al., J. Gen Virol., 36:59, 1977); human fibrosarcoma cell line (e.g., HT1080); baby hamster kidney cells (BHK21, ATCC CCL 10); Chinese hamster ovary cells +/−DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

In some embodiments, the present invention provides recombinant follistatin proteins or recombinant follistatin-Fc fusion proteins produced from non-human cells or human cells. In some embodiments, the present invention provides recombinant follistatin proteins or recombinant follistatin-Fc fusion proteins produced from CHO cells or HT1080 cells.

Typically, cells that are engineered to express a recombinant follistatin protein or a recombinant follistatin-Fc fusion protein may comprise a transgene that encodes a recombinant follistatin protein or recombinant follistatin-Fc fusion protein described herein. It should be appreciated that the nucleic acids encoding a recombinant follistatin protein or recombinant follistatin-Fc fusion protein may contain regulatory sequences, gene control sequences, promoters, non-coding sequences and/or other appropriate sequences for expressing the recombinant follistatin protein or recombinant follistatin-Fc fusion protein. Typically, the coding region is operably linked with one or more of these nucleic acid components.

The coding region of a transgene may include one or more silent mutations to optimize codon usage for a particular cell type. For example, the codons of a follistatin transgene may be optimized for expression in a vertebrate cell. In some embodiments, the codons of a follistatin transgene may be optimized for expression in a mammalian cell, for example a CHO cell. In some embodiments, the codons of a follistatin transgene may be optimized for expression in a human cell.

### Pharmaceutical Composition and Administration

The present invention further provides pharmaceutical compositions comprising therapeutically active ingredients in accordance with the invention (e.g., recombinant follistatin protein, or recombinant follistatin-Fc fusion protein), together with one or more pharmaceutically acceptable carrier or excipient. Such pharmaceutical compositions may optionally comprise one or more additional therapeutically-active substances.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

Formulations of the pharmaceutical compositions described herein may be prepared by any method known or hereafter developed in the art of pharmacology. In general, such preparatory methods include the step of bringing the active ingredient into association with a diluent or another excipient or carrier and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient or carrier, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations may additionally comprise a pharmaceutically acceptable excipient or carrier, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium or carrier is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient or carrier is at least 95%, at least 96%, at least 97%, at least 98%, at least 99%, or 100% pure. In some embodiments, an excipient or carrier is approved for use in humans and for veterinary use. In some embodiments, an excipient or carrier is approved by United States Food and Drug Administration. In some embodiments, an excipient or carrier is pharmaceutical grade. In some embodiments, an excipient or carrier meets the standards of the United States Pharmacopoeia (USP), the European Pharmacopoeia (EP), the British Pharmacopoeia, and/or the International Pharmacopoeia.

Pharmaceutically acceptable excipients or carriers used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients or carriers may optionally be included in pharmaceutical formulations. Excipients or carriers such as cocoa butter and suppository waxes, coloring agents, coating agents, sweetening, flavoring, and/or perfuming agents can be present in the composition, according to the judgment of the formulator.

Suitable pharmaceutically acceptable excipients or carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interfere with their activity. In a preferred embodiment, a water-soluble carrier suitable for intravenous administration is used.

A suitable pharmaceutical composition or medicament, if desired, can also contain minor amounts of wetting or emulsifying agents, or pH buffering agents. A composition can be a liquid solution, suspension, emulsion, tablet, pill, capsule, sustained release formulation, or powder. A composition can also be formulated as a suppository, with traditional binders and carriers such as triglycerides. Oral formulations can include standard carriers such as pharmaceutical grades of mannitol, lactose, starch, magnesium stearate, polyvinyl pyrollidone, sodium saccharine, cellulose, magnesium carbonate, etc.

A pharmaceutical composition or medicament can be formulated in accordance with the routine procedures as a pharmaceutical composition adapted for administration to human beings. For example, in some embodiments, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

A recombinant follistatin protein or recombinant follistatin-Fc fusion protein described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### Routes of Administration

A recombinant follistatin protein or recombinant follistatin-Fc fusion protein described herein (or a composition or medicament containing a recombinant follistatin protein described herein) is administered by any appropriate route. In some embodiments, a recombinant follistatin protein, recombinant follistatin-Fc fusion protein or a pharmaceutical composition containing the same is administered systemically. Systemic administration may be intravenous, intradermal, inhalation, transdermal (topical), intraocular, intramuscular, subcutaneous, intramuscular, oral and/or transmucosal administration. In some embodiments, a recombinant follistatin protein, recombinant follistatin-Fc fusion protein or a pharmaceutical composition containing the same is administered subcutaneously. As used herein, the term “subcutaneous tissue”, is defined as a layer of loose, irregular connective tissue immediately beneath the skin. For example, the subcutaneous administration may be performed by injecting a composition into areas including, but not limited to, the thigh region, abdominal region, gluteal region, or scapular region. In some embodiments, a recombinant follistatin protein, recombinant follistatin-Fc fusion protein or a pharmaceutical composition containing the same is administered intravenously. In some embodiments, a recombinant follistatin protein, recombinant follistatin-Fc fusion protein or a pharmaceutical composition containing the same is administered orally. In some embodiments, a recombinant follistatin protein, recombinant follistatin-Fc fusion protein or a pharmaceutical composition containing the same is administered intramuscularly. For example, the intramuscular administration may be performed by injecting a composition into areas including, but not limited to, a muscle of the thigh region, abdominal region, gluteal region, scapular region, or to any muscle disclosed in Table 1. More than one route can be used concurrently, if desired.

In some embodiments, administration results only in a localized effect in an individual, while in other embodiments, administration results in effects throughout multiple portions of an individual, for example, systemic effects. Typically, administration results in delivery of a recombinant follistatin protein or recombinant follistatin-Fc fusion protein to one or more target tissues. In some embodiments, the recombinant follistatin protein or recombinant follistatin-Fc fusion protein is delivered to one or more target tissues including, but not limited to, heart, brain, spinal cord, striated muscle (e.g., skeletal muscle), smooth muscle, kidney, liver, lung, and/or spleen. In some embodiments, the recombinant follistatin protein or recombinant follistatin-Fc fusion protein is delivered to the heart. In some embodiments, the recombinant follistatin protein or recombinant follistatin-Fc fusion protein is delivered to striated muscle, in particular, skeletal muscle. In some embodiments, the recombinant follistatin protein or recombinant follistatin-Fc fusion protein is delivered to triceps, tibialis anterior, soleus, gastrocnemius, biceps, trapezius, deltoids, quadriceps, and/or diaphragm.

Dosage Forms and Dosing Regimen

In some embodiments, a composition is administered in a therapeutically effective amount and/or according to a dosing regimen that is correlated with a particular desired outcome (e.g., with treating or reducing risk for a muscular dystrophy, such as Duchenne muscular dystrophy).

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, severity of cardiac defect and/or level of risk of cardiac defect, etc., or combinations thereof). Such doses or amounts can be determined by those of ordinary skill. In some embodiments, an appropriate dose or amount is determined in accordance with standard clinical techniques. Alternatively or additionally, in some embodiments, an appropriate dose or amount is determined through use of one or more in vitro or in vivo assays to help identify desirable or optimal dosage ranges or amounts to be administered.

In various embodiments, a recombinant follistatin protein is administered at a therapeutically effective amount. Generally, a therapeutically effective amount is sufficient to achieve a meaningful benefit to the subject (e.g., treating, modulating, curing, preventing and/or ameliorating the underlying disease or condition). In some particular embodiments, appropriate doses or amounts to be administered may be extrapolated from dose-response curves derived from in vitro or animal model test systems.

In some embodiments, a provided composition is provided as a pharmaceutical formulation. In some embodiments, a pharmaceutical formulation is or comprises a unit dose amount for administration in accordance with a dosing regimen correlated with achievement of the reduced incidence or risk of a muscular dystrophy, such as Duchenne muscular dystrophy.

In some embodiments, a formulation comprising a recombinant follistatin protein or recombinant follistatin-Fc fusion protein described herein administered as a single dose. In some embodiments, a formulation comprising a recombinant follistatin protein or recombinant follistatin-Fc fusion protein described herein is administered at regular intervals. Administration at an “interval,” as used herein, indicates that the therapeutically effective amount is administered periodically (as distinguished from a one-time dose). The interval can be determined by standard clinical techniques. In some embodiments, a formulation comprising a recombinant follistatin protein or recombinant follistatin-Fc fusion protein described herein is administered bimonthly, monthly, twice monthly, triweekly, biweekly, weekly, twice weekly, thrice weekly, daily, twice daily, or every six hours. The administration interval for a single individual need not be a fixed interval, but can be varied over time, depending on the needs of the individual.

As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day.

In some embodiments, a formulation comprising a recombinant follistatin protein or recombinant follistatin-Fc fusion protein described herein is administered at regular intervals indefinitely. In some embodiments, a formulation comprising a recombinant follistatin protein or recombinant follistatin-Fc fusion protein described herein is administered at regular intervals for a defined period.

As described herein, the term “therapeutically effective amount” is largely determined based on the total amount of the therapeutic agent contained in the pharmaceutical compositions of the present invention. A therapeutically effective amount is commonly administered in a dosing regimen that may comprise multiple unit doses. For any particular composition, a therapeutically effective amount (and/or an appropriate unit dose within an effective dosing regimen) may vary, for example, depending on route of administration or on combination with other pharmaceutical agents.

In some embodiments, administration of a recombinant follistatin protein or recombinant follistatin-Fc fusion protein reduces the intensity, severity, or frequency, or delays the onset of at least one DMD sign or symptom. In some embodiments administration of a recombinant follistatin protein or recombinant follistatin-Fc fusion protein reduces the intensity, severity, or frequency, or delays the onset of at least one DMD sign or symptom selected from the group consisting of muscle wasting, skeletal deformation, cardiomyopathy, muscle ischemia, cognitive impairment, and impaired respiratory function.

In some embodiments, administration of a recombinant follistatin protein or recombinant follistatin-Fc fusion protein improves clinical outcome as measured by a 6 minute walk test, quantitative muscle strength test, timed motor performance test. Brooke and Vignos limb function scales, pulmonary function test (forced vital capacity, forced expiratory volume in 1 second, peak expiratory flow rate, maximal inspiratory and expiratory pressures), health-related quality of life, knee and elbow flexors, elbow extensors, shoulder abduction, grip strength, time to rise from supine position, North Start Ambulatory Assessment, timed 10 meter walk/run, Egen-Klassification scale, Gowers score, Hammersmith motor ability, hand held myometry, range of motion, goniometry, hypercapnia, Nayley Scales of Infant and Toddler Development, and/or a caregiver burden scale.

### Combination Therapy

In some embodiments, a recombinant follistatin protein is administered in combination with one or more known therapeutic agents (e.g., corticosteroids) currently used for treatment of a muscular dystrophy. In some embodiments, the known therapeutic agent(s) is/are administered according to its standard or approved dosing regimen and/or schedule. In some embodiments, the known therapeutic agent(s) is/are administered according to a regimen that is altered as compared with its standard or approved dosing regimen and/or schedule. In some embodiments, such an altered regimen differs from the standard or approved dosing regimen in that one or more unit doses is altered (e.g., reduced or increased) in amount, and/or in that dosing is altered in frequency (e.g., in that one or more intervals between unit doses is expanded, resulting in lower frequency, or is reduced, resulting in higher frequency).

In some embodiments, a recombinant follistatin protein or recombinant follistatin-Fc fusion protein is administered in combination with one or more additional therapeutic agents. In one embodiment the additional therapeutic agent is a corticosteroid, e.g., prednisone. In another embodiment, the additional therapeutic agent is a glucocorticoid, e.g., deflazacort. In another embodiment, the additional therapeutic agent is an anti-Flt-1 antibody or antigen binding fragment thereof. In another embodiment the additional therapeutic agent is an RNA modulating therapeutic. The RNA modulating therapeutic may be an exon-skipping therapeutic or gene therapy. The RNA modulating therapeutic may be, for example, Drispersen, CAT-1004, FG3019, PRO044, PRO045, Eteplirsen (AVI-4658), SRP-4053, SRP-4045, SRP-4050, SRP-4044, SRP-4052, SRP-4055 or SRP-4008. In some embodiments the additional therapeutic agent is currently used for treatment of a muscular dystrophy. In other embodiments the additional therapeutic agent may also be used to treat other diseases or disorders. In some embodiments, the known therapeutic agent(s) is/are administered according to its standard or approved dosing regimen and/or schedule. In some embodiments, the known therapeutic agent(s) is/are administered according to a regimen that is altered as compared with its standard or approved dosing regimen and/or schedule. In some embodiments, such an altered regimen differs from the standard or approved dosing regimen in that one or more unit doses is altered (e.g., reduced or increased) in amount, and/or in that dosing is altered in frequency (e.g., in that one or more intervals between unit doses is expanded, resulting in lower frequency, or is reduced, resulting in higher frequency).

### EXAMPLES

### Example 1. Follistatin-Fc Fusion Proteins Target Myostatin

This example illustrates follistatin-Fc fusion protein binding to target and non-target ligands. Without wishing to be bound by theory, it is contemplated that activation of Smad2/3 pathway by myostatin and activin A leads to inhibition of myogenic protein expression and as a result, myoblasts do not differentiate into muscle. Therefore, myostatin and activin A are considered viable targets for stimulation of muscle regeneration. However, many myostatin and activin A antagonists such as soluble activin receptor type IIB (sActRIIB) also bind bone morphogenetic proteins (BMPs) due to certain structural similarities. BMPs, especially, BMP-9 and BMP-10, are considered pivotal morphogenetic signals, orchestrating tissue architecture throughout the body. Inhibition of such BMPs may lead to undesired pathological conditions. Follistatin also binds to cell surface heparan-sulfate proteoglycans through a basic heparin-binding sequence (HBS) in the first of three FS domains. Without wishing to be bound by theory, inactivation, reduction or modulation of heparin binding by, e.g., mutation or deletion of the HBS may increase in vivo exposure and/or half-life of follistatin and/or follistatin fusion proteins. As described in detail below, the experimental data described in this example confirm that follistatin-Fc fusion proteins specifically target myostatin with high affinity and do not bind to non-target BMPs or heparin with meaningful affinity.

Specifically, the binding affinity (K_{D}) and kinetics of follistatin-Fc fusion proteins for myostatin, activin A, heparin, BMP-9 and BMP-10 were assessed using BIAcore® assays and standard methods as described below.

To determine binding affinity and kinetics for myostatin, anti-humanFc (GE catalog # BR-1008-39) was immobilized onto two flow cells CM5 chip for 420 seconds at a flow rate of 10 μl/min. The running buffer was HBS-EP+. All samples and controls were diluted to 10 μg/mL using the running buffer. Myostatin (0.1 mg/mL in 4 mM HCl) (R&D Systems, Catalogue number 788-G8-010/CF) was diluted to 0.3125, 0.625, 1.25, 2.5 and 5 nM based on molecular weight of 25 kDa. The assay was performed with a capture setting of 8 seconds at a flow rate of 50 μL/min, association for 300 seconds at a flow rate of 50 μL/min and dissociation for 1200 seconds at a flow rate of 50 μL/min, followed by regeneration using 3M MgCl₂ for 30 seconds at a flow rate of 60 μL/min.

To determine binding affinity and kinetics for Activin A anti-humanFc (GE catalog # BR-1008-39) was immobilized onto two flow cells CM5 chip for 420 seconds at a flow rate of 10 μl/min. The running buffer was HBS-EP+. All samples and controls were diluted to 10 μg/mL using the running buffer. Activin A (0.1 mg/mL in 4 mM HCl) (R&D Systems, Catalog number 338-AC-050 CF) was diluted to 0.156, 0.3125, 0.625, 1.25, and 2.5 nM using the molecular weight of 26 kDa.

To determine binding affinity and kinetics for heparin, biotinylated heparin was prepared on the day of the assay at 1 mg/mL then diluted to 100 μg/mL in HBS+N. Streptavidin chip flow cells were prepared by immobilization for 5 minutes at 5 μl/min at 100 μg/mL using HBS+N buffer. Samples were diluted in HBS+EP to a concentration of 0.31 nM to 25 nM. The assay was performed using an association time of 300 seconds at a flow rate of 30 μL/min and a dissociation time of 300 seconds followed by regeneration with 4M NaCl for 30 seconds, immediately followed by second regeneration with 4M NaCl for 30 seconds.

To determine binding affinity and kinetics for BMP-9 and/or BMP-10, anti-human Fc was coupled to FC3 and FC4 at approximately 6000 to 9000 RU on a CM5 chip. The ActRIIB-Fc protein was used as a positive control (R&D Systems, Catalogue number 339-RBB-100) for binding to BMP-9 and BMP-10. For analysis of BMP-9 binding, all samples were diluted to 2.5 μg/mL and the running buffer was HBS+EP. For analysis of BMP-10 binding, all samples were diluted to 5 μg/mL and the running buffer was HBS+EP+0.5 mg/mL BSA. Analysis conditions include a contact time of 180 seconds, a dissociation time of 300 seconds and a flow rate of 30 μL/minute. BMP-9 (R&D Systems, Catalogue number 3209-BP-010CF) and BMP-10 (R&D Systems, Catalogue number 2926-BP-025CF) were diluted in three fold serial dilutions from 25 nM to 0.19 nM. Exemplary results are shown in Table 8.

TABLE 8
Exemplary Binding Affinity and Kinetics Data

| | Myostatin | Activin A | Heparin | | |
|---|---|---|---|---|---|
| | Binding | Binding | Binding | | |
| | K_{D} (pM) | K_{D} (pM) | K_{D} (nM) | BMP-9 | BMP-10 |
| | Range | Range | Range | Range | Range |
| Follistatin-Fc | tested 5- | tested 2.5- | tested 25- | tested 25- | tested 25- |
| Fusion Protein | 0.31 nM | 0.15 nM | 0.31 nM | 0.190 nM | 0.190 nM |
| FS315WT-hFc | 6-10 | 1-2 | 0.3 | no binding | no binding in |
| | | | | in range | range tested |
| FS315WT- | 20.2 | not tested | 0.16 | not tested | not tested |
| hFcLALA | | | | | |
| ActRIIB-Fc | not tested | not tested | not tested | 0.44 | 0.9 |
| FS315K(81, 82)A- | 11.9 | not tested | 1.50 | no binding in | no binding in |
| hFcLALA | | | | range tested | range tested |
| FS315K(81, 82)A- | 10.7 | not tested | 1.30 | not tested | not tested |
| GGG- hFcLALA | | | | | |
| FS315K(76, 81, 82)A - | 11.3 | not tested | 9.40 | no binding in | no binding in |
| hFcLALA | | | | range tested | range tested |
| FS303K(76, 81, 82)A - | 12.7 | not tested | 0.57 | no binding in | no binding in |
| hFcLALA | | | | range tested | range tested |
| FS315K(76, 81, 82)A - | 10.9 | not tested | 3.70 | not tested | not tested |
| GGG- hFcLALA | | | | | |
| FS303K(76, 81, 82)A - | 11.6 | not tested | 0.51 | not tested | not tested |
| GGG- hFcLALA | | | | | |
| FS315K82T- | 15.0 | not tested | 1.40 | no binding in | no binding in |
| hFcLALA | | | | range tested | range tested |
| FS303K82T- | 9.7 | not tested | 0.33 | no binding in | no binding in |
| hFcLALA | | | | range tested | range tested |
| FS315K82T-GGG- | 13.0 | not tested | 1.30 | not tested | not tested |
| hFcLALA | | | | | |
| FS303K82T-GGG- | 9.6 | not tested | 0.18 | not tested | not tested |
| hFcLALA | | | | | |
| FS315K82E- | 11.90 | not tested | 1.50 | not tested | not tested |
| hFcLALA | | | | | |
| FS315K(75, 76)E- | 11.70 | not tested | 1.10 | not tested | not tested |
| hFcLALA | | | | | |
| FS315K(76, 81)E- | 11 | not tested | 4 | not tested | not tested |
| hFcLALA | | | | | |
| FS315K(76, 82)E- | 10.50 | not tested | 4 | not tested | no binding in |
| hFcLALA | | | | | range tested |
| FS315K(81, 82)E- | 9.87 | not tested | 11 | not tested | no binding in |
| hFcLALA | | | | | range tested |
| FS315K(81, 82)D- | 7.09 | 0.47 | 20.6 | no binding in | no binding in |
| hFcLALA | | | | range tested | range tested |
| FS315K(76, 81, 82)E- | 2-6 | 1-2 | >25 | no binding in | no binding in |
| hFcLALA | | | | range tested | range tested |
| FS315K(76, 81, 82)D - | 5.92 | 0.76 | >25 | no binding in | no binding in |
| hFcLALA | | | | range tested | range tested |
| FS315K(76, 81, 82)E/ | 4.5 | not tested | >25 | not tested | not tested |
| V88E-hFcLALA | | | | | |
| FS315(del75-86)- | 57.10 | not tested | >25 | not tested | no binding in |
| hFcLALA | | | | | range tested |
| FS315R86N/V88T- | 12.70 | not tested | 1.30-1.7 | not tested | no binding in |
| hFcLALA | | | | | range tested |
| FS315K75N/C77T/ | 40.30 | not tested | 1 4 | not tested | no binding in |
| K82T- hFcLALA | | | | | range tested |
| FS315R78N/N80T- | 13.00 | not tested | 0.85 | not tested | not tested |
| hFcLALA | | | | | |
| FS315P85T- | 12.40 | not tested | 0.37 | not tested | not tested |
| hFcLALA | | | | | |
| FS315C66A/K75N/ | 24.4 | not tested | 3fold less than | no binding in | no binding in |
| C77T-hFcLALA | | | FS315wt-hFc | range tested | range tested |
| FS315C66S/K75N/ | 6.4 | not tested | 3fold less than | no binding in | no binding in |
| C77T-hFcLALA | | | FS315wt-hFc | range tested | range tested |
| FS315K(76, 81, 82)E- | 14.8 | not tested | >25 | not tested | not tested |
| mFc | | | | | |
| MONOVALENT | | | | | |
| MOLECULES: | | | | | |
| monoFS315wt- | 2.89 | not tested | 29.2 | not tested | not tested |
| hFcLALA | | | | | |
| monoFS315ΔHBS- | 3.3 | not tested | >25 | not tested | not tested |
| hFcLALA | | | | | |
| monoFS315K(76, 81, | <4 | not tested | >25 | no binding in | no binding in |
| 82)E-hFcLALA | | | | range tested | range tested |

As shown in Table 8, follistatin fusion proteins bind myostatin with high affinity but do not bind BMP-9 and/or BMP-10. In studies testing follistatin-Fc fusion protein binding to BMP-10, no kinetic constants were determined in the range tested (25000 to 190 pM). This represents a binding affinity approximately 430 times higher than the weakest myostatin binding K_{D}. In studies testing follistatin-Fc fusion protein binding to BMP-9, no kinetic constants were determined in the range tested (25000 to 190 pM). This represents a binding affinity approximately 1400 times higher than the weakest myostatin binding K_{D}.

### Example 2. Follistatin-Fc Fusion Protein Binding to the FcRn Receptor

Some mutations in the Fc domain lead to reduced binding with the FcRn receptor and thereby have reduced in vivo serum half-life. The binding affinity of follistatin-Fc fusion proteins to the FcRn receptor was assessed using standard methods. Exemplary results are shown in Table 9.

TABLE 9
Exemplary FcRn Binding Data

| Follistatin-Fc fusion protein | KD (nM) |
|---|---|
| FS315WT-hFc | 114.0 |
| FS315K(81, 82)A-hFcLALA | 107.0 |
| FS315K(76, 81, 82)A-hFcLALA | 86.5 |
| FS315K(76, 82)E-hFcLALA | 125.0 |
| FS315K(81, 82)E-hFcLALA | 178.0 |
| FS315K(81, 82)D-hFcLALA | 24.7 |
| FS315K(76, 81, 82)E-hFcLALA | 96-131 |
| FS315K(76, 81, 82)D-hFcLALA | 59.9 |
| FS315ΔHBS-hFcLALA | 372.0 |
| FS315(del75-86)-hFcLALA | 126.0 |
| FS315K82T--hFcLALA | 44.8 |
| FS303K82T-hFcLALA | 27.6 |
| FS315R86N/V88T-hFcLALA | 69.5 |
| FS315K75N/C77T/K82T-hFcLALA | 126.0 |
| FS315C66A/K75N/C77T-hFcLALA | 28.0 |
| FS315C66S/K75N/C77T-hFcLALA | 83.0 |
| monoFS315K(76, 81, 82)E-hFcLALA | 40.6 |
| monoFS315-hFcLALA | 12.8 |
| monoFS315ΔHBS-hFcLALA | 36.7 |

Some mutations in the Fc domain lead to reduced binding with the Fc Gamma IA receptor and thereby have reduced effector function. The binding affinity of follistatin-Fc fusion proteins to the Fc Gamma IA receptor was assessed using standard methods. The binding affinity of follistatin-Fc fusion proteins to the Fc Gamma IA receptor was assessed using standard methods.

To determine binding affinities for Fc gamma receptor IA, Follistatin-Fc proteins were diluted in sodium acetate pH 5.0 to 2.5 μg/mL and immobilized on CM5 chip at ^{˜}150 RU. Fc Gamma Receptor RIA was purchased as lyophilized stock from R&D Systems, Catalog #1257-FC-050. For analysis of Fc Gamma receptor IA the running buffer was HBS-P+. Analysis conditions include a contact time of 180 seconds, a dissociation time of 600 seconds and a flow rate of 30 μL/minute. Regeneration conditions were 10 mM sodium phosphate pH 2.5, 500 mM NaCl for 10 sec at 304/min with 30 sec stability. Fc Gamma Receptor IA was diluted 62.5 nM-0.49 nM. Exemplary results are shown in Table 10.

TABLE 10
Exemplary Fc Gamma IA Binding Data

| Follistatin-Fc fusion protein | Fc gamma IA K_{D} (nM) |
|---|---|
| FS315wt-hFc (comparator protein) | 0.14 |
| FS315K(76, 81, 82)E-hFcLALA | 81.9 |
| FS315K(76, 81, 82)D-hFcLALA | 58.8 |

### Example 3. Follistatin-Fc Fusion Proteins have Extended Serum Half-Life

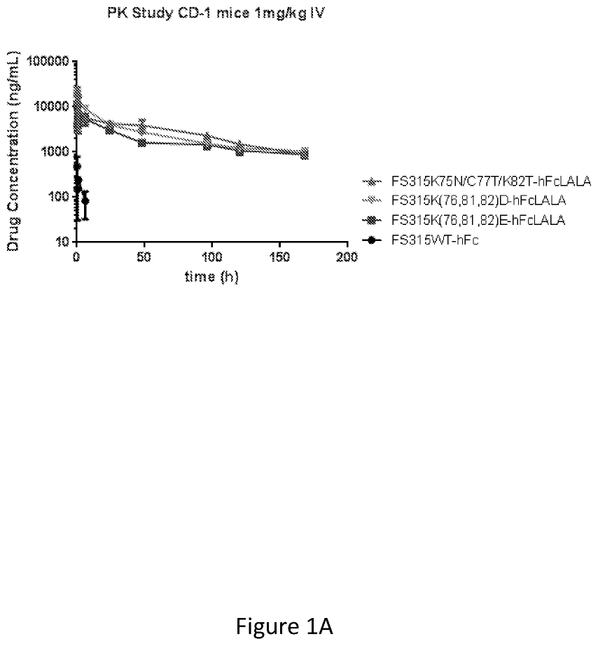

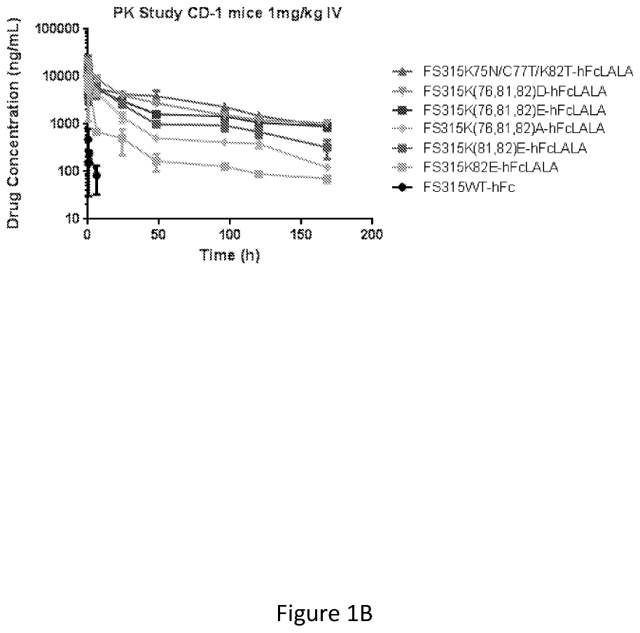

Follistatin is reported to have a short serum half-life. For example, typical commercial FS315 protein has a serum half-life of about an hour. In this example, the in vivo half-life of follistatin-Fc fusion proteins comprising the various mutations as shown in FIG. 1A, FIG. 1B and Table 11 were determined to have significantly extended serum half-lives as compared to a comparator protein.

Specifically, CD-1 mice were administered the individual follistatin-Fc fusion proteins intravenously at the doses indicated in Table 11. Following administration, serum levels of follistatin-Fc fusion protein were collected at various time points (FIG. 1A and FIG. 1B). The serum half-life of the recombinant follistatin-Fc fusion proteins ranged from 45.7 to 194 hours.

TABLE 11
Exemplary follistatin-Fc fusion protein in vivo PK data

| | Dose | T_{1/2} | AUC_{INF} | %AUC_{Extrapolated} | Cl | Vₛₛ |
|---|---|---|---|---|---|---|
| Fusion protein | (mg/kg) | (hr) | (hr*ng/ml) | (%) | (mL/hr/kg) | (mL/kg) |
| WT (Comparator | 1.0 | 3.77 | 1550 | 29.3 | 322 | 1490 |
| fusion protein) | | | | | | |
| K82E | 1.0 | 93.5 | 67700 | 14.1 | 11.8 | 807 |
| K(76, 82)E | 1.0 | 95.4 | 262000 | 17.4 | 2.67 | 222 |
| K(82, 84)E | 1.0 | 64.2 | 336000 | 9.97 | 2.98 | 167 |
| K(81, 82)E | 1.0 | 104 | 236500 | 17.6 | 4.20 | 418 |
| K(81, 82)D | 1.0 | 194 | 529000 | 37.7 | 1.89 | 371 |
| R78E/K82E | 1.0 | 94.5 | 760000 | 25.0 | 1.32 | 151 |
| K(76, 81, 82)A | 1.0 | 60.4 | 179000 | 5.95 | 4.46 | 205 |
| K(76, 81, 82)E | 1.0 | 116 | 646000 | 28.8 | 1.86 | 253 |
| K(76, 81, 82)D | 1.0 | 85.4 | 598000 | 21.7 | 1.67 | 168 |
| K(76, 82, 84)E | 1.0 | 74.7 | 638000 | 17.3 | 1.57 | 136 |
| K(76, 81, 82)E/V88E | 1.0 | 87.9 | 993000 | 26.1 | 1.01 | 122 |
| R78E/K(82, 84)E | 1.0 | 71.5 | 453000 | 18.0 | 2.21 | 202 |
| K75N/C77T/K82T | 1.0 | 55.6 | 566000 | 12.7 | 0.886 | 71.0 |
| G74N/K76T/P85T | 1.0 | 45.7 | 92100 | 5.54 | 10.9 | 509 |
| C66A/K75N/C77T | 1.0 | 51.6 | 331000 | 9.37 | 3.02 | 194 |

### Example 4. Follistatin-Fc Fusion Proteins Inhibit Myostatin and Activin A

The ability of follistatin-Fc fusion proteins to inhibit myostatin and activin A activity was tested using a luciferase gene reporter assay. Rhabdomyosarcoma A204 cells were stably transfected with the pGL3(CAGA)12-Luc plasmid, which contains a Smad3-selective response element in front of the firefly luciferase gene. 1.2 nM myostatin or activin A was used for stimulation of Smad3 signaling. Fusion proteins were incubated with either myostatin or activin A for 30 minutes at room temperature prior to addition to cells, and then after 24 hours of incubation at 37° C. luciferase activity was measured. The concentration of myostatin or activin A used for the signaling assays was 1.2 nM. As shown in Table 14, the follistatin-Fc fusion proteins inhibited myostatin in a stimulation assay with IC₅₀s ranging from less than 0.5 nM to over 1.5 nM. As shown in Table 12, the follistatin-Fc fusion proteins inhibited activin A in a stimulation assay with IC₅₀s ranging from less than 0.5 nM to over 1.5 nM.

TABLE 12
IC50s for myostatin and activin A from
cell-based assay for SMAD pathway

| | Myostatin | Activin A |
|---|---|---|
| Sample Name | IC50 (nM) | IC50 (nM) |
| FS315wt-hFc (comparator protein) | 0.4 | 0.7 |
| ActRIIB-Fc | 0.5 | 0.5 |
| FS315ΔHBS-hFcLALA | 0.4 | Not tested |
| FS315K(76, 81, 82)A-hFcLALA | 0.3 | 0.7 |
| FS315K82E--hFcLALA | 0.4 | 0.6 |
| FS315K(81, 82)E-hFcLALA | 0.6 | 1.0 |
| FS315K(82, 84)E-hFcLALA | 0.7 | 1.1 |
| FS315K(76, 81, 82)E-hFcLALA | 0.5 | 0.7 |
| FS315K(81, 82, 84)E-hFcLALA | 0.4 | 1.0 |
| FS315K(76, 81, 82)E/V88E-hFcLALA | 0.6 | 0.6 |
| FS315R78E/K82E-hFcLALA | 0.5 | 1.0 |
| FS315R78E/K(82, 84)E-hFcLALA | 0.5 | 0.6 |
| FS315K75N/C77T/K82T-hFcLALA | 0.7 | 1.1 |
| FS315G74N/K76T/P85T-hFcLALA | 1.0 | 1.3 |
| FS315C66S/K75N/C77T-hFcLALA | 1.5 | 2.1 |
| FS315K(76, 81, 82)E-mFc | 0.7 | 0.8 |

### Example 5. In Vivo Efficacy of Follistatin-Fc Fusion Proteins-Systemic Administration

This example demonstrates that systemic administration of follistatin-Fc fusion proteins (e.g., FS315K(76,81,82)E-hFcLALA, FS315K(76,81,82)E-mFc) to wild-type mice and the mdx mouse model of Duchenne muscular dystrophy results in a trend of increased muscle mass in vivo at a dose of 10 mg/kg administered either intravenously or subcutaneously.

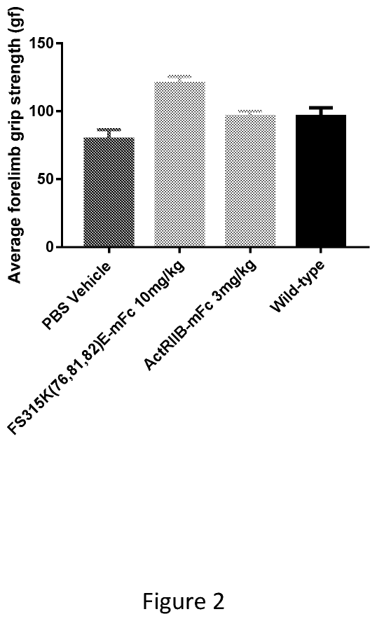

Specifically in one study male C57BL/6 (wild-type mice) were administered vehicle (i.e., PBS) or FS315K(76,81,82)E-hFcLALA by intravenous injection at a dose of 10 mg/kg or subcutaneous injection at a dose of 20 mg/kg twice a week for 4 weeks. In a second study male mdx mice were administered vehicle (i.e., PBS) or FS315K(76,81,82)E-mFc by subcutaneous injection at a dose of 10 mg/kg or the mouse soluble activin receptor type IIB chimeric Fc fusion (ActRIIB-mFc) by subcutaneous injection at a dose of 3 mg/kg twice a week for 12 weeks. Twenty-four hours after the last treatment, the mice were sacrificed and the gastrocnemius and quadriceps muscles were collected and weighed. Exemplary data in Table 13 show that there was a significant increase in the weight of the gastrocnemius and quadriceps muscles from both mdx and C57BL/6 mice as compared to the gastrocnemius or quadriceps muscles treated with vehicle alone. Thus, there is a clear indication that recombinant follistatin-Fc fusion proteins increase muscle mass when dosed systemically in wild-type mice and in an animal model of DMD. In the mdx study, forelimb grip strength was measured after 11 weeks of dosing. Exemplary data in FIG. 2 shows that there was a significant increase in forelimb grip strength of mdx mice treated with FS315K(76,81,82)E-mFc compared to the grip strength of animals treated with vehicle alone. The magnitude of grip strength for the FS315K(76,81,82)E-mFc treated animals was greater than animals treated with the ActRIIB-mFc positive control and also greater than wild-type C57BL/10ScSnJ animals.

TABLE 13
Muscle Mass Data (% Change Relative to Vehicle) from the C57BL/6 and mdx Mouse

| | | % change over vehicle | | | |
|---|---|---|---|---|---|
| | | C57BL/6 | | mdx | |
| | | Gastroc. | Quad. | Gastroc. | Quad. |
| FS315K(76, 81, 82)E-hFcLALA | 10 mg/kg (IV 2x weekly) | +28% | +33% | | |
| FS315K(76, 81, 82)E-hFcLALA | 20 mg/kg (SC 2x weekly) | +32% | +39% | | |
| FS315K(76, 81, 82)E-mFc | 10 mg/kg (SC 2x weekly) | | | 31% | 36% |
| ActRIIB-mFc | 3 mg/kg (SC 2x weekly) | | | 24% | 28% |

### Example 6: Characterization of Follistatin Constructs

This example demonstrates that systemic administration of follistatin-Fc fusion proteins (e.g., FS315K(76,81,82)E-hFcLALA, FS315K(76,81,82)E-mFc) to wild-type mice and the mdx mouse model of Duchenne muscular dystrophy results in a trend of increased muscle mass in vivo at a dose of 10 mg/kg administered either intravenously or subcutaneously.

Changes in pI were also assessed for follistatin-Fc fusion proteins. Table 14 below shows a shift to a more acidic pI with E and D mutations in the HBS as well as with hyperglycosylation variants. The shift in pI correlates with decreased heparin binding and increased in vivo exposure.

The cIEF profile (pI range) was determined using a NanoPro Instrument (ProteinSimple). The final Protein concentration tested was 0.0025 mg/ml, 124 loaded in the well. The dilution buffer used was DPBS and Urea/Chaps (10M/0.6%). Additional reagents used included G2 premix: 4-9 (ProteinSimple 040-969), pI standard ladder 1 (ProteinSimple 040-644), primary antibody: rabbit anti-FS pAB (Abcam # ab47941) at 1:100 dilution, secondary antibody: rabbit anti-IgG HRP conjugate (Promega #4011) at 1:100 dilution, and substrate: Luminol/Peroxide XDR.

TABLE 14
Isoelectic Point (pI) Ranges in Follistatin-Fc Fusion Proteins

| Follistatin-Fc fusion protein | pI range |
|---|---|
| FS315WT-hFc | 5.51-6.17 |
| FSΔHBS-hFcLALA | 4.82-5.72 |
| FSΔHBS-GGG-hFcLALA | 4.82-5.72 |
| FS315del75-86-hFcLALA | 4.83-5.26 |
| FS315K(81, 82)A-hFcLALA | 5.31-5.96 |
| FS315K(81, 82)A-GGG-hFcLALA | 5.23-5.93 |
| FS315K(76, 81, 82)A-hFcLALA | 5.24-5.93 |
| FS303K(76, 81, 82)A-hFcLALA | 5.28-5.93 |
| FS315K(76, 81, 82)A-GGG-hFcLALA | 5.23-5.87 |
| FS303K(76, 81, 82)A-GGG-hFcLALA | 5.23-5.93 |
| FS315K82T-hFcLALA | 5.29-5.93 |
| FS303K82T-hFcLALA | 5.27-6.14 |
| FS315K82T-GGG-hFcLALA | 5.48-5.95 |
| FS303K82T-GGG-hFcLALA | 5.23-6.15 |
| FS315K82E-hFcLALA | 5.48-6.09 |
| FS315K(75, 76)E-hFcLALA | 5.05-5.26 |
| FS315K(76, 82)E-hFcLALA | 4.89-5.26 |
| FS315K(81, 82)E-hFcLALA | 4.83-5.25 |
| FS315K(81, 82)D-hFcLALA | 4.88-5.59 |
| FS315K(76, 81, 82)E-hFcLALA | 4.87-5.80 |
| FS315K(76, 81, 82)D-hFcLALA | 4.82-5.67 |
| FS315P85T-hFcLALA | 5.51-6.09 |
| FS315R86N/V88T-hFcLALA | 5.49-6.08 |
| FS315K75N/C77T/K82T-hFcLALA | 4.89-5.26 |
| FS315R78N/N80T-hFcLALA | 5.47-6.09 |
| FS315C66A/K75N/C77T-hFcLALA | 4.81-6.47 |
| FS315C66S/K75N/C77T-hFcLALA | 4.82-6.59 |
| FS315K(76, 81, 82)E-mFc | 4.7-5.3 |
| MonoFS315K(76, 81, 82)E-hFcLALA | 4.7-5.3 |
| MonoFS315WT-hFcLALA | 4.7-5.67 |
| MonoFS315ΔHBS-hFcLALA | 4.83-5.9 |

In summary, the above examples demonstrate that recombinant follistatin-Fc fusion proteins are highly effective in inducing muscle hypertrophy in a DMD disease model by, for example, systemic administration. Muscle hypertrophy in the mdx mouse model translated to functional improvement in forelimb grip strength. Thus, recombinant follistatin-Fc fusion proteins can be effective protein therapeutics for the treatment of DMD.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

We claim:

1. A recombinant follistatin polypeptide comprising an amino acid sequence at least 80% identical to SEQ ID NO: 1, SEQ ID NO: 2, SEQ ID NO: 3, SEQ ID NO: 4, or SEQ ID NO: 5, wherein the recombinant follistatin protein has a heparin binding sequence (HBS), and wherein one or more amino acids within the HBS is substituted with an amino acid having a less positive charge in comparison to the substituted amino acid.

2. The recombinant follistatin polypeptide of claim 1, wherein the one or more amino acids within the HBS are substituted with an amino acid having a neutral charge.

3. The recombinant follistatin polypeptide of claim 1, wherein the one or more amino acids within the HBS are substituted with an amino acid having a negative charge.

4. The recombinant follistatin polypeptide of any one of claims 1-3, wherein the one or more comprises at least 1, 2, 3, 4, 5, 6, 7, 8, 9, or 10 amino acids.

5. The recombinant follistatin polypeptide of claim 4, wherein the one or more comprises 3 amino acids.

6. The recombinant follistatin polypeptide of any one of claims 1-5, wherein the recombinant polypeptide has decreased heparin binding affinity in comparison to naturally occurring follistatin.

7. The recombinant follistatin polypeptide of any one of claims 1-6, wherein the recombinant follistatin protein does not bind to BMP-9 or BMP-10.

8. The recombinant follistatin polypeptide of claim 1, wherein the recombinant follistatin protein has a sequence at least 80% identical to any one of SEQ ID NO: 12-40 or SEQ ID NO: 101-106.

9. The recombinant follistatin polypeptide of any one of claims 1-8, comprising an amino acid sequence at least 80% identical to SEQ ID NO:2, SEQ NO:4 or SEQ ID NO:5 and
wherein the amino acids corresponding to positions 66 to 88 of SEQ ID NO:2, SEQ NO:4 or SEQ ID NO:5 are identical to any one of SEQ ID NO:42-67 or SEQ ID NO:111-116.

10. The recombinant follistatin polypeptide of claim 9, wherein the amino acid sequence corresponding to positions 66 to 88 of SEQ ID NO: 2, SEQ ID NO: 4, or SEQ ID NO: 5 are identical to any one of SEQ ID NO: 58-67 or SEQ ID NO: 111-113.

11. The recombinant follistatin polypeptide of claim 10, wherein the recombinant follistatin polypeptide is a hyperglycosylation mutant.

12. A recombinant follistatin polypeptide comprising an amino acid sequence at least 80% identical to SEQ ID NO:2, SEQ ID NO:4 or SEQ ID NO:5 and
comprising any one of the amino acid variations selected from the group consisting of C66S, C66A, G74N, K75E, K75N, K76A, K76D, K765, K76E, C77S, C77T, R78E, R78N, N80T, K81A, K81D, K82A, K82D, K81E, K82T, K82E, K84E, P85T, R86N, V88E and V88T, or combinations thereof.

13. The recombinant follistatin polypeptide of claim 9 or 12, wherein the amino acid sequence is at least 90% identical to SEQ ID NO:2, SEQ NO:4 or SEQ ID NO:5.

14. The recombinant follistatin polypeptide of claim 9 or 12, wherein the amino acid sequence is at least 95% identical to SEQ ID NO:2, SEQ NO:4 or SEQ ID NO:5.

15. The recombinant follistatin polypeptide of claim 9 or 12, wherein the amino acid sequence is at least 98% identical to SEQ ID NO:2, SEQ NO:4 or SEQ ID NO:5.

16. The recombinant follistatin polypeptide of claim 9 or 12, wherein the amino acid sequence is 100% identical to SEQ ID NO:2, SEQ NO:4 or SEQ ID NO:5.

17. A recombinant follistatin polypeptide comprising an amino acid sequence selected from the group consisting of SEQ NO:12, SEQ ID NO:17-30 and SEQ ID NO:32-40.

18. A recombinant follistatin fusion protein comprising a follistatin polypeptide of any one of claims 1-17 and an IgG Fc domain.

19. A recombinant follistatin fusion protein comprising a follistatin polypeptide and a human IgG Fc domain,
wherein the recombinant follistatin polypeptide comprises an amino acid sequence at least 80% identical to SEQ ID NO:2, SEQ ID NO:4 or SEQ ID NO:5 and
wherein the amino acids corresponding to positions 66 to 88 of SEQ ID NO:2, SEQ ID NO:4 or SEQ ID NO:5 are identical to SEQ ID NO:41, 42, 43 or 58.

20. The recombinant follistatin fusion protein of claim 19, wherein the recombinant follistatin polypeptide comprises an amino acid sequence that is at least 90% identical to SEQ ID NO:2, SEQ ID NO:4 or SEQ ID NO:5.

21. The recombinant follistatin fusion protein of claim 19, wherein the recombinant follistatin polypeptide comprises an amino acid sequence that is at least 95% identical to SEQ ID NO:2, SEQ ID NO:4 or SEQ ID NO:5.

22. The recombinant follistatin fusion protein of claim 19, wherein the recombinant follistatin polypeptide comprises an amino acid sequence that is at least 98% identical to SEQ ID NO:2, SEQ ID NO:4 or SEQ ID NO:5.

23. The recombinant follistatin fusion protein of claim 19, wherein the recombinant follistatin polpypeptide comprises an amino acid sequence that is 100% identical to SEQ ID NO:2, SEQ ID NO:4 or SEQ ID NO:5.

24. A recombinant follistatin fusion protein comprising a follistatin polypeptide and an IgG Fc domain,
wherein the follistatin polypeptide comprises an amino acid sequence selected from any one of the group consisting of SEQ ID NO:12, SEQ ID NO:13, and SEQ ID NO:15 to SEQ ID NO:40.

25. The recombinant follistatin fusion protein of any one of claims 18-24, wherein the IgG Fc domain comprises an amino acid substitution; and
wherein the amino acid substitution is selected from the group consisting of L234A, L235A, H433K, N434F, and combinations thereof, according to EU numbering.

26. The recombinant follistatin fusion protein of any one of claims 18-24, wherein the IgG Fc domain comprises an amino acid sequence of SEQ ID NO:6 and
wherein the amino acid sequence comprises an amino acid substitution selected from the group consisting of L234A, L235A, H433K, N434F, and combinations thereof, according to EU numbering.

27. The recombinant follistatin fusion protein of any one of claims 18-24, wherein the IgG Fc domain comprises an amino acid sequence selected from the group consisting of SEQ ID NO:7 to SEQ ID NO:11.

28. The recombinant follistatin fusion protein of any one of claims 18-25, wherein the IgG Fc domain is a human IgG Fc domain.

29. The recombinant follistatin fusion protein of any one of claims 18-25, wherein the IgG Fc domain is an IgG1, IgG2, IgG3 or IgG4 Fc domain.

30. A recombinant follistatin fusion protein comprising an amino acid sequence of any one of SEQ ID NO:73 to SEQ ID NO:100, SEQ ID NO: 117, or SEQ ID NO: 118.

31. The recombinant follistatin fusion protein of any one of claims 18-30, wherein the protein binds to myostatin with an affinity dissociation constant (K_{D}) of 1 to 100 pM.

32. The recombinant follistatin fusion protein of any one of claims 18-30, wherein the protein binds to activin A with an affinity dissociation constant (K_{D}) of 1 to 100 pM.

33. The recombinant follistatin fusion protein of any one of claims 18-30, wherein the protein does not bind to bone morphogenic protein-9 (BMP-9) and/or bone morphogenic protein-10 (BMP-10) in the range of 0.2 nM to 25 nM.

34. The recombinant follistatin fusion protein of any one of claims 18-30, wherein the protein binds to heparin with an affinity dissociation constant (K_{D}) of 0.1 to >25 nM.

35. The recombinant follistatin fusion protein of any one of claims 18-30, wherein the protein binds to the FcRn receptor with an affinity dissociation constant (K_{D}) of 25 to 400 nM.

36. The recombinant follistatin fusion protein of any one of claims 18-30, wherein protein inhibits myostatin at an IC₅₀ of 0.1 to 10 nM.

37. The recombinant follistatin fusion protein of any one of claims 18-30, wherein protein inhibits activin A at an IC₅₀ of 0.1 to 10 nM.

38. A pharmaceutical composition comprising the recombinant follistatin fusion protein of any one of claims 18-37 and a pharmaceutically acceptable carrier.

39. A polynucleotide comprising a nucleotide sequence encoding the recombinant follistatin polypeptide of any one of claims 1-17.

40. A polynucleotide comprising a nucleotide sequence encoding the recombinant follistatin fusion protein of any one of claims 18-30.

41. An expression vector comprising the polynucleotide of claim 39 or 40.

42. A host cell comprising a polynucleotide of claim 39 or 40 or an expression vector of claim 30.

43. A method of making a recombinant follistatin fusion protein that specifically binds to myostatin comprising culturing the host cell of claim 42.

44. A hybridoma cell producing a recombinant follistatin polypeptide of any one of claims 1-17 or a recombinant follistatin fusion protein of any one of claims 18-30.

45. A method of treating Duchenne Muscular Dystrophy (DMD), the method comprising:
administering to a subject who is suffering from or susceptible to DMD an effective amount of the recombinant follistatin fusion protein of any one of claims 18-37 or the pharmaceutical composition of claim 38, such that at least one symptom or feature of DMD is reduced in intensity, severity, or frequency, or has delayed onset.

46. The method of claim 45, wherein the method further comprises administering to the subject one or more additional therapeutic agents.

47. The method of claim 46, wherein the one or more additional therapeutic agents are selected from the group consisting of an anti-Flt-1 antibody or fragment thereof, edasalonexent, pamrevlumab, prednisone, deflazacort, RNA modulating therapeutics, exon-skipping therapeutics and gene therapy.

48. The method of any one of claims 45-47, wherein an effective amount of the recombinant follistatin fusion protein is administered parenterally.

49. The method of claim 48, wherein the parenteral administration is selected from the group consisting of intravenous, intradermal, intrathecal, inhalation, transdermal (topical), intraocular, intramuscular, subcutaneous, transmucosal administration, or combinations thereof.

50. The method of claim 49, wherein the parenteral administration is intravenous administration.

51. The method of claim 49, wherein the parenteral administration is subcutaneous administration.

52. The method of any one of claims 45-51, wherein the recombinant follistatin fusion protein is administered daily, twice weekly, weekly, monthly or bimonthly.

53. The method of claim 52, wherein the recombinant follistatin fusion protein is administered twice weekly.

54. The method of any one of claims 45-53, wherein the recombinant follistatin fusion protein is delivered to one or more skeletal muscles selected from Table 1.

55. The method of any one of claims 45-54, wherein the administration of the recombinant follistatin fusion protein results in an increase in the mass of a muscle relative to a control.

56. The method of claim 55, wherein the muscle is one or more skeletal muscles selected from Table 1.

57. The method of claim 56, wherein the muscle is selected from the group consisting of diaphragm, triceps, soleus, tibialis anterior, gastrocnemius, extensor digitorum longus, rectus abdominus, quadriceps, and combinations thereof.

58. The method of claim 57, wherein the muscle is the gastrocnemius muscle.

59. The method of any one of claims 55-58, wherein the increase in the mass of the muscle is an increase of at least 10%, 20%, 30%, 40%, 50%, 60%, 70%, 80%, 90%, 100%, 150%, 200% or 500% relative to a control.

60. The method of any one of claims 45-59, wherein the administration of the recombinant follistatin fusion protein results in muscle regeneration, increased muscle strength, increased flexibility, increased range of motion, increased stamina, reduced fatigability, increased blood flow, improved cognition, improved pulmonary function, inflammation inhibition, reduced muscle fibrosis, and/or reduced muscle necrosis.

61. The method of any one of claims 45-59, wherein the at least one symptom or feature of DMD is selected from the group consisting of muscle wasting, muscle weakness, muscle fragility, muscle necrosis, muscle fibrosis, joint contracture, skeletal deformation, cardiomyopathy, impaired swallowing, impaired bowel and bladder function, muscle ischemia, cognitive impairment, behavioral dysfunction, socialization impairment, scoliosis, and impaired respiratory function.

62. A method for inhibiting myostatin in a subject, the method comprising administering to the muscle of a subject a composition comprising an effective amount of the recombinant follistatin fusion protein of any one of claims 18-30.


SEQUENCE LISTING (121 sequences)

SEQ ID NO: 1 - protein, 344 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 2 - protein, 315 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 3 - protein, 332 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQ

SEQ ID NO: 4 - protein, 303 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQ

SEQ ID NO: 5 - protein, 288 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 6 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, Artificial Sequence
  source: /note=“Description of Artificial Sequence: Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, Artificial Sequence
  source: /note=“Description of Artificial Sequence: Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 9 - protein, 227 aa, Artificial Sequence
  source: /note=“Description of Artificial Sequence: Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 10 - protein, 232 aa, Artificial Sequence
  source: /note=“Description of Artificial Sequence: Synthetic polypeptide”
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 226 aa, Artificial Sequence [sequence omitted]

SEQ ID NO: 12 - protein, 303 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 13 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGQSCVVDQTGSPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 14 - protein, 10 aa, Artificial Sequence
  source: /note=“Description of Artificial Sequence: Synthetic peptide”
QSCVVDQTGS

SEQ ID NO: 15 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 16 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 17 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKENKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 18 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGEECRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 19 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNKENKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 20 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNEENKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 21 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNEENKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 22 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNEENKPRCECAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 23 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKKNEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 24 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNKKNEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 25 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKENEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 26 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCEMNKKNEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 27 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNKENEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 28 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCEMNKENKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 29 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCEMNKENEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 30 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNEKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 31 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKTNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 32 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKKNKTRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 33 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCNMTKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 34 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKKNKPNCTCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 35 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGNKTRMNKTNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 36 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPNKSCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 37 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPNKTCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 38 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPNKTCRMNKKNKTRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 39 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETSENVDCGPGNKTRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 40 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETAENVDCGPGNKTRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 41 - protein, 23 aa, Artificial Sequence
  source: /note=“Description of Artificial Sequence: Synthetic peptide”
CENVDCGPGQSCVVDQTGSPRCV

SEQ ID NO: 42 - protein, 23 aa, Artificial Sequence
  source: /note=“Description of Artificial Sequence: Synthetic peptide”
CENVDCGPGKKCRMNAANKPRCV

SEQ ID NO: 43 - protein, 23 aa, Artificial Sequence
  source: /note=“Description of Artificial Sequence: Synthetic peptide”
CENVDCGPGKACRMNAANKPRCV

SEQ ID NO: 44 - protein, 23 aa, Artificial Sequence
  source: /note=“Description of Artificial Sequence: Synthetic peptide”
CENVDCGPGKKCRMNKENKPRCV

SEQ ID NO: 45 - protein, 23 aa, Artificial Sequence
  source: /note=“Description of Artificial Sequence: Synthetic peptide”
CENVDCGPGEECRMNKKNKPRCV

SEQ ID NO: 46 - protein, 23 aa, Artificial Sequence
  source: /note=“Description of Artificial Sequence: Synthetic peptide”
CENVDCGPGKECRMNKENKPRCV

SEQ ID NO: 47 - protein, 23 aa, Artificial Sequence
  source: /note=“Description of Artificial Sequence: Synthetic peptide”
CENVDCGPGKKCRMNEENKPRCV

SEQ ID NO: 48 - protein, 23 aa, Artificial Sequence
  source: /note=“Description of Artificial Sequence: Synthetic peptide”
CENVDCGPGKECRMNEENKPRCV

SEQ ID NO: 49 - protein, 23 aa, Artificial Sequence
  source: /note=“Description of Artificial Sequence: Synthetic peptide”
CENVDCGPGKECRMNEENKPRCE

SEQ ID NO: 50 - protein, 23 aa, Artificial Sequence
  source: /note=“Description of Artificial Sequence: Synthetic peptide”
CENVDCGPGKKCRMNKKNEPRCV

SEQ ID NO: 51 - protein, 23 aa, Artificial Sequence
  source: /note=“Description of Artificial Sequence: Synthetic peptide”
CENVDCGPGKECRMNKKNEPRCV

SEQ ID NO: 52 - protein, 23 aa, Artificial Sequence
  source: /note=“Description of Artificial Sequence: Synthetic peptide”
CENVDCGPGKKCRMNKENEPRCV

SEQ ID NO: 53 - protein, 23 aa, Artificial Sequence
  source: /note=“Description of Artificial Sequence: Synthetic peptide”
CENVDCGPGKKCEMNKKNEPRCV

SEQ ID NO: 54 - protein, 23 aa, Artificial Sequence
  source: /note=“Description of Artificial Sequence: Synthetic peptide”
CENVDCGPGKECRMNKENEPRCV

SEQ ID NO: 55 - protein, 23 aa, Artificial Sequence
  source: /note=“Description of Artificial Sequence: Synthetic peptide”
CENVDCGPGKKCEMNKENKPRCV

SEQ ID NO: 56 - protein, 23 aa, Artificial Sequence
  source: /note=“Description of Artificial Sequence: Synthetic peptide”
CENVDCGPGKKCEMNKENEPRCV

SEQ ID NO: 57 - protein, 23 aa, Artificial Sequence
  source: /note=“Description of Artificial Sequence: Synthetic peptide”
CENVDCGPGKECRMNEKNKPRCV

SEQ ID NO: 58 - protein, 23 aa, Artificial Sequence
  source: /note=“Description of Artificial Sequence: Synthetic peptide”
CENVDCGPGKKCRMNKTNKPRCV

SEQ ID NO: 59 - protein, 23 aa, Artificial Sequence
  source: /note=“Description of Artificial Sequence: Synthetic peptide”
CENVDCGPGKKCRMNKKNKTRCV

SEQ ID NO: 60 - protein, 23 aa, Artificial Sequence
  source: /note=“Description of Artificial Sequence: Synthetic peptide”
CENVDCGPGKKCNMTKKNKPRCV

SEQ ID NO: 61 - protein, 23 aa, Artificial Sequence
  source: /note=“Description of Artificial Sequence: Synthetic peptide”
CENVDCGPGKKCRMNKKNKPNCT

SEQ ID NO: 62 - protein, 23 aa, Artificial Sequence
  source: /note=“Description of Artificial Sequence: Synthetic peptide”
CENVDCGPGNKTRMNKTNKPRCV

SEQ ID NO: 63 - protein, 23 aa, Artificial Sequence
  source: /note=“Description of Artificial Sequence: Synthetic peptide”
CENVDCGPNKSCRMNKKNKPRCV

SEQ ID NO: 64 - protein, 23 aa, Artificial Sequence
  source: /note=“Description of Artificial Sequence: Synthetic peptide”
CENVDCGPNKTCRMNKKNKPRCV

SEQ ID NO: 65 - protein, 23 aa, Artificial Sequence
  source: /note=“Description of Artificial Sequence: Synthetic peptide”
CENVDCGPNKTCRMNKKNKTRCV

SEQ ID NO: 66 - protein, 23 aa, Artificial Sequence
  source: /note=“Description of Artificial Sequence: Synthetic peptide”
SENVDCGPGNKTRMNKKNKPRCV

SEQ ID NO: 67 - protein, 23 aa, Artificial Sequence
  source: /note=“Description of Artificial Sequence: Synthetic peptide”
AENVDCGPGNKTRMNKKNKPRCV

SEQ ID NO: 68 - protein, 9 aa, Artificial Sequence
  source: /note=“Description of Artificial Sequence: Synthetic peptide”
ALEVLFQGP

SEQ ID NO: 69 - protein, 3 aa, Artificial Sequence
  source: /note=“Description of Artificial Sequence: Synthetic peptide”
GGG

SEQ ID NO: 70 - protein, 21 aa, Artificial Sequence
  source: /note=“Description of Artificial Sequence: Synthetic peptide”
GAPGGGGGAAAAAGGGGGGAP

SEQ ID NO: 71 - protein, 39 aa, Artificial Sequence
  source: /note=“Description of Artificial Sequence: Synthetic polypeptide”
GAPGGGGGAAAAAGGGGGGAPGGGGGAAAAAGGGGGGAP

SEQ ID NO: 72 - protein, 57 aa, Artificial Sequence
  source: /note=“Description of Artificial Sequence: Synthetic polypeptide”
GAPGGGGGAAAAAGGGGGGAPGGGGGAAAAAGGGGGGAPGGGGGAAAAAGGGGGGAP

SEQ ID NO: 73 - protein, 530 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGQSCVVDQTGSPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKENKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGEECRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNKENKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNEENKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNEENKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNEENKPRCECAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKKNEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNKKNEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKENEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCEMNKKNEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNKENEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCEMNKENKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCEMNKENEPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKECRMNEKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKTNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKKNKTRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCNMTKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNKKNKPNCTCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGNKTRMNKTNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPNKSCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPNKTCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPNKTCRMNKKNKTRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETSENVDCGPGNKTRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETAENVDCGPGNKTRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGNKSRMNKTNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 102 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETSENVDCGPGNKSRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 103 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETAENVDCGPGNKSRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 104 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNDDNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 105 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKDCRMNDDNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 106 - protein, 315 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKDCRMNKDNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 107 - protein, 23 aa, Homo sapiens
CENVDCGPGKKCRMNKKNKPRCV

SEQ ID NO: 108 - protein, 23 aa, Artificial Sequence
  source: /note=“Description of Artificial Sequence: Synthetic peptide”
CENVDCGPGSTCVVDQTNNAYCV

SEQ ID NO: 109 - protein, 11 aa, Artificial Sequence
  source: /note=“Description of Artificial Sequence: Synthetic peptide”
CENVDCGPGCV

SEQ ID NO: 110 - skipped

SEQ ID NO: 111 - protein, 23 aa, Artificial Sequence
  source: /note=“Description of Artificial Sequence: Synthetic peptide”
CENVDCGPGNKSRMNKTNKPRCV

SEQ ID NO: 112 - protein, 23 aa, Artificial Sequence
  source: /note=“Description of Artificial Sequence: Synthetic peptide”
SENVDCGPGNKSRMNKKNKPRCV

SEQ ID NO: 113 - protein, 23 aa, Artificial Sequence
  source: /note=“Description of Artificial Sequence: Synthetic peptide”
AENVDCGPGNKSRMNKKNKPRCV

SEQ ID NO: 114 - protein, 23 aa, Artificial Sequence
  source: /note=“Description of Artificial Sequence: Synthetic peptide”
CENVDCGPGKKCRMNDDNKPRCV

SEQ ID NO: 115 - protein, 23 aa, Artificial Sequence
  source: /note=“Description of Artificial Sequence: Synthetic peptide”
CENVDCGPGKDCRMNDDNKPRCV

SEQ ID NO: 116 - protein, 23 aa, Artificial Sequence
  source: /note=“Description of Artificial Sequence: Synthetic peptide”
CENVDCGPGKDCRMNKDNKPRCV

SEQ ID NO: 117 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKDCRMNKDNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKDCRMNDDNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 317 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 120 - protein, 542 aa, Artificial Sequence
  source: /note=“Description of Artificial Sequence: Synthetic polypeptide”
GNCWLRQAKNGRCQVLYKTELSKEECCSTGRLSTSWTEEDVNDNTLFKWMIFNGGAPNCIPCKETCENVDCGPGKKCRMNDDNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 12 aa, Homo sapiens
KKCRMNKKNKPR